SEQUENCE LISTING (20 sequences)

SEQ ID NO: 1 - protein, 17 aa, Homo sapiens
MWLQSLLLLGTVACSIS

SEQ ID NO: 2 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 3 - protein, 12 aa, synthetic construct
ESKYGPPCPPCP

SEQ ID NO: 4 - protein, 110 aa, synthetic construct
APEFLGGPSVFLFPPKPKDTLMISRTPEVTCVVVDVSQEDPEVQFNWYVDGVEVHNAKTKPREEQFNSTYRVVRVLTVLHQDWLNGKEYKCKVSNKGLPSSIEKTISKAK

SEQ ID NO: 5 - protein, 107 aa, synthetic construct
GQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSPGK

SEQ ID NO: 6 - protein, 22 aa, synthetic construct
MALIVLGGVAGLLLFIGLGIFF

SEQ ID NO: 7 - protein, 420 aa, synthetic construct
MWLQSLLLLGTVACSISDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHESKYGPPCPPCPAPEFLGGPSVFLFPPKPKDTLMISRTPEVTCVVVDVSQEDPEVQFNWYVDGVEVHNAKTKPREEQFNSTYRVVRVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSPGKMALIVLGGVAGLLLFIGLGIFF

SEQ ID NO: 8 - DNA, 456 bp, Homo sapiens
  source 1..456: mol_type = other DNA
gattgcgatatcgaaggcaaagatggtaagcagtacgaaagtgttctgatggtttccattgaccaactgcttgactccatgaaggaaattggttctaattgtcttaataatgagtttaacttttttaagagacacatttgcgatgccaataaggagggaatgttccttttccgcgccgcaagaaagctgaggcaattcttgaagatgaactccaccggcgatttcgacttgcaccttttgaaggtcagcgaaggaacgaccattttgctgaactgcaccggccaggtgaagggtcggaagccagcggcactgggagaggcgcagccgactaagagccttgaggaaaataagtctcttaaagaacagaaaaaacttaacgacctgtgttttctgaaacggttgcttcaggaaatcaagacctgttggaacaaaatactgatggggactaaagagcat

SEQ ID NO: 9 - protein, 581 aa, synthetic construct
MWLQSLLLLGTVACSISHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFCVRCRHRR

SEQ ID NO: 10 - protein, 581 aa, synthetic construct
MWLQSLLLLGTVACSISDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFCVRCRHRR

SEQ ID NO: 11 - protein, 586 aa, synthetic construct
MWLQSLLLLGTVACSISYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFCVRCRHRR

SEQ ID NO: 12 - protein, 586 aa, synthetic construct
MWLQSLLLLGTVACSISHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFCVRCRHRR

SEQ ID NO: 13 - DNA, 1767 bp, synthetic construct
  source 1..1767: mol_type = other DNA
gctagcgccaccatgtggctgcagagcctgctgctcttgggcactgtggcctgcagcatctctcacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccggcggcggcggcagcggcggcggcggcagcggcggcggcggcagcgattgcgatatcgaaggcaaagatggtaagcagtacgaaagtgttctgatggtttccattgaccaactgcttgactccatgaaggaaattggttctaattgtcttaataatgagtttaacttttttaagagacacatttgcgatgccaataaggagggaatgttccttttccgcgccgcaagaaagctgaggcaattcttgaagatgaactccaccggcgatttcgacttgcaccttttgaaggtcagcgaaggaacgaccattttgctgaactgcaccggccaggtgaagggtcggaagccagcggcactgggagaggcgcagccgactaagagccttgaggaaaataagtctcttaaagaacagaaaaaacttaacgacctgtgttttctgaaacggttgcttcaggaaatcaagacctgttggaacaaaatactgatggggactaaagagcatgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggtaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaatggccctgattgtgctggggggcgtcgccggcctcctgcttttcattgggctaggcatcttcttctgtgtaaggtgcaggcacaggagataataagtcgac

SEQ ID NO: 14 - DNA, 1767 bp, synthetic construct
  source 1..1767: mol_type = other DNA
gctagcgccaccatgtggctgcagagcctgctgctcttgggcactgtggcctgcagcatctctgattgcgatatcgaaggcaaagatggtaagcagtacgaaagtgttctgatggtttccattgaccaactgcttgactccatgaaggaaattggttctaattgtcttaataatgagtttaacttttttaagagacacatttgcgatgccaataaggagggaatgttccttttccgcgccgcaagaaagctgaggcaattcttgaagatgaactccaccggcgatttcgacttgcaccttttgaaggtcagcgaaggaacgaccattttgctgaactgcaccggccaggtgaagggtcggaagccagcggcactgggagaggcgcagccgactaagagccttgaggaaaataagtctcttaaagaacagaaaaaacttaacgacctgtgttttctgaaacggttgcttcaggaaatcaagacctgttggaacaaaatactgatggggactaaagagcatggcggcggcggcagcggcggcggcggcagcggcggcggcggcagccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggtaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaatggccctgattgtgctggggggcgtcgccggcctcctgcttttcattgggctaggcatcttcttctgtgtaaggtgcaggcacaggagataataagtcgac

SEQ ID NO: 15 - DNA, 1767 bp, synthetic construct; identical to SEQ ID NO: 13
  source 1..1767: mol_type = other DNA

SEQ ID NO: 16 - DNA, 1782 bp, synthetic construct
  source 1..1782: mol_type = other DNA
gctagcgccaccatgtggctgcagagcctgctgctcttgggcactgtggcctgcagcatctctcacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccggcggcggcggcagcggcggcggcggcagcggcggcggcggcagctactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggtaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaatggccctgattgtgctggggggcgtcgccggcctcctgcttttcattgggctaggcatcttcttctgtgtaaggtgcaggcacaggagataataagtcgac

SEQ ID NO: 17 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 18 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 15 aa, synthetic construct
GGGGSGGGGSGGGGS

SEQ ID NO: 20 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0158164 A1
(43) Pub. Date: Jun. 11, 2026

(54) METHODS AND COMPOSITIONS COMPRISING MEMBRANE BOUND CYTOKINES

(71) Applicant: The Trustees of the University of Pennsylvania, Philadelphia, PA (US)

(72) Inventors: James L. Riley, Downingtown, PA (US); Mosha Deng, Philadelphia, PA (US); Jonah Walter Perelman, Randolph, NJ (US)

(21) Appl. No.: 19/388,638

(22) Filed: Nov. 13, 2025

Related U.S. Application Data
(60) Provisional application No. 63/720,477, filed on Nov. 14, 2024.

(51) Int. Cl. A61K 47/69

(52) U.S. Cl. CPC A61K 47/6901

(57) ABSTRACT

The present disclosure generally relates to compositions and methods comprising membrane-bound cytokines, including combinations thereof. In some instances, the polypeptides comprise IL-7, IL-21, and/or IL-18.

### CROSS-REFERENCE TO RELATED APPLICATION

The present application is entitled to priority under 35 U.S.C. § 119(e) to U.S. Provisional Patent Application No. 63/720,477 filed on Nov. 14, 2024, which is herein incorporated by reference in its entirety.

### STATEMENT REGARDING FEDERALLY SPONSORED RESEARCH

This invention was made with government support under AI149680 awarded by the National Institutes of Health. The government has certain rights in the invention.

### REFERENCE TO AN ELECTRONIC SEQUENCE LISTING

The Sequence Listing submitted herewith as a xml file named “046483-7442xx.xml,” created on Nov. 12, 2025 and having a size of 29,447 bytes, is herein incorporated by reference in its entirety.

### BACKGROUND

Immunotherapy involving the priming and expansion of T lymphocytes (T cells) holds the promise of effective treatment of cancer and infectious diseases. Current clinical uses of adoptive transfer therapy in patients with cancer and viral infections regularly involves the infusion of T cells that have been stimulated, cloned and expanded for many weeks in vitro on autologous dendritic cells (DC), virally infected B cells, and/or allogeneic feeder cells. However, adoptive T cell immunotherapy clinical trials routinely require billions of cells per patient. In order to produce these quantities of cells, T cells must be subjected to many fold expansion in vitro, requiring up to 40 population doublings. Furthermore, for optimal engraftment potential and possible therapeutic benefit at the time of re-infusion, it is important to ensure that the expanded T cells remain functional and are not senescent or exhausted.

Methods of expanding T cell clones and/or lines for adoptive immunotherapy have proven to have certain drawbacks. The standard culture of pure CD8⁺ cells is limited by apoptosis, diminution of biological function and/or proliferation, and obtaining a sufficient number of cells to be useful has been particularly difficult. Indeed, it is possible that such T cells that are currently infused into patients, may have a limited replicative capacity, and therefore, could not stably engraft to provide long-term protection from disease. Furthermore, the various techniques available for expanding human T cells have relied primarily on the use of accessory cells (i.e. cells that support or promote T cell survival and proliferation such as PBMCs, DCs, B cells, monocytes, etc.) and/or exogenous growth factors, such as interleukin-2 (IL-2), IL-7, and IL-15. The requirement for accessory cells presents a significant problem for long-term culture systems because these cells are relatively short-lived. Therefore, in a long-term culture system, APCs must be continually obtained and replenished. The necessity for a renewable supply of accessory cells is problematic for treatment of immunodeficiencies in which accessory cells are affected. Likewise, the need for exogenous mixtures of cytokines to support expansion and function of cultured T cells adds considerably to the costs, especially when stable sources of GMP-grade materials are required.

A need exists to provide means to stimulate T cells to combat various acute and chronic diseases and to promulgate sufficient numbers of therapeutic T cells for adoptive immunotherapy. The present invention addresses this need.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 presents the sequence of membrane-bound IL-7 (mIL-7) (SEQ ID NO: 6) with various segments in accordance with one embodiment. These segments include: human GM-CSF signal peptide (SEQ ID NO: 1); mature human IL-7 variant 1 long isoform (SEQ ID NO: 2); Mod IgG4 hinge region (SEQ ID NO: 3); IgG4 CH2 domain (SEQ ID NO: 4); IgG4 CH3 domain (SEQ ID NO: 5); and Hu CD4 Tm Domain (SEQ ID NO: 6).

FIGS. 2A-2B present graphical representations of results related to mIL-7 expression. FIG. 2A presents a graphical representation of K562 wild type cells that were stained for IL-7. FIG. 2B presents a graphical representation of K562 cells transduced with mIL-7 that were stained for IL-7.

FIG. 3 presents a schematic representation of a dual, membrane bound construct comprising IL-21 and IL-7 referred to as IL21GS7 (SEQ ID NO: 9).

FIG. 4 presents a schematic representation of a dual, membrane bound construct comprising IL-7 and IL-21 referred to as IL7GS21 (SEQ ID NO: 10).

FIG. 5 presents a schematic representation of a dual, membrane bound construct comprising IL-18 and IL-21 referred to as IL18GS21 (SEQ ID NO: 11).

FIG. 6 presents a schematic representation of a dual, membrane bound construct comprising IL-21 and IL-18 referred to as IL21GS18 (SEQ ID NO: 12).

FIGS. 7A-7B present graphical representations of pSTAT3 and pSTAT5 assays which analyzed the effects of mIL-7.

FIG. 8A-FIG. 8B present graphical representations of pSTAT3 and pSTAT5 assays analyzing the effects of mIL-7 on STAT3 and STAT5 activation.

FIG. 9 depicts combokines containing IL-7 or IL-18 fused to IL-21 through a (G4S)3 linker in various configurations were tested to identify the optimal sequence. The proximal cytokine is fused to the IgG4 hinge and CH2/3 domains and CD4 transmembrane domain to enable membrane tethering.

FIGS. 10A-10B: Detection of combokine expression. Histogram (FIG. 10A) and dot plot (FIG. 10B) views showing expression of IL-7, IL-18, or IL-21 by fluorescence-activated cell sorting (FACS).

FIGS. 11A-11B: Combokine-mediated induction of STAT3, STAT5, and NF-kB signaling. (A) Induction of STAT3 and STAT5 signaling by IL7-G4S-IL21 and IL21-G4S-IL7 combokines. (B) Induction of STAT3, STAT5 and NF-κB signaling by IL18-G4S-IL21 and IL21-G4S-IL18 combokines.

FIGS. 12A-12C: Combokine-mediated induction of STAT3 or STAT5 using T cells from three independent donors (Donor 1 (FIG. 12A), Donor 2 (FIG. 12B) or Donor 3 (FIG. 12C)) stimulated with combokine-transformed K562 cells.

### DETAILED DESCRIPTION

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, protein and nucleic acid chemistry, and nucleic acid hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

That the disclosure may be more readily understood, select terms are defined below.

As used herein, the terms “a,” “an,” or “the” are used to include one or more than one unless the context clearly dictates otherwise. By way of example, “an element” means one element or more than one element. The term “or” is used to refer to a nonexclusive “or” unless otherwise indicated. The statement “at least one of A and B” or “at least one of A or B” has the same meaning as “A, B, or A and B.”

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 and so forth, as well as individual numbers within that range, for example, 1, 2, 2.7, 3, 4, 5, 5.3, and 6. This applies regardless of the breadth of the range.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, the term “autologous” is meant to refer to any material derived from the same individual to which it is later to be re-introduced into the individual.

As used herein, the term “combokine” is used with reference to a chimeric membrane-bound polypeptide comprising functional domains from at least two different cytokines.

As used herein, the term “construct” is used with reference to a membrane-bound cytokine or combokine as described herein, or a nucleic acid or vector encoding the membrane-bound cytokine or combokine.

“Co-stimulatory ligand”, as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to CD86, 4-1BBL, BTLA and a Toll ligand receptor.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “therapeutic” as used herein means a treatment and/or prophylaxis. A therapeutic effect is obtained by any degree of suppression, remission, or eradication of a disease state.

To “treat” a disease as the term is used herein, means to reduce the frequency or severity of at least one sign or symptom of a disease or disorder experienced by a subject.

As used herein, to “alleviate” a disease means reducing the severity of one or more symptoms of the disease.

The terms “patient”, “subject”, and “individual” are used interchangeably and are intended to include living organisms that may be subjected to treatment for a given disease, e.g., mammals. A “subject”, “patient”, or “individual”, as used herein, can be a human or non-human mammal. Non-human mammals include, for example, livestock and pets, such as ovine, bovine, porcine, canine, feline, and murine mammals, as well as simian and non-human primate mammals. Preferably, the subject is human.

As used herein, the terms “effective amount” and “therapeutically effective amount” are used interchangeably and refer to the amount required to reduce or improve at least one symptom or change in a clinical marker of a disease relative to an untreated patient. The effective amount of the treatment used for therapeutic treatment of the disease varies depending upon the manner of the specific disorder, condition or disease, extent of the disorder, condition or disease, and administration of the cells, as well as the age, body weight, and general health of the subject. The effective amount is capable of achieving a particular desired biological result and/or provides a therapeutic or prophylactic benefit.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “expression” as used herein is defined as the transcription and/or translation of a particular nucleotide sequence driven by its promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the terms “conservative variation” or “conservative substitution” generally refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

As used herein, the term “isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

The term “membrane insertion moiety” is used with reference to an amino acid sequence that directs the insertion or functional contact of the membrane-bound polypeptide with a membrane.

The term “recombinant polypeptide” as used herein is defined as a polypeptide produced by using recombinant DNA methods.

The term “recombinant DNA” as used herein is defined as DNA produced by joining pieces of DNA from different sources.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

By the term “modified” as used herein, is meant a changed state or structure of a molecule or cell of the invention. Molecules may be modified in many ways, including chemically, structurally, and functionally. Cells may be modified through the introduction of nucleic acids.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain.

In some embodiments, the TCR may be modified on any cell comprising a TCR, including, for example, a helper T cell, a cytotoxic T cell, a memory T cell, regulatory T cell, natural killer T cell, and gamma delta T cell.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “oligonucleotide” typically refers to short polynucleotides. It will be understood that when a nucleotide sequence is represented by a DNA sequence (i.e., A, 5 T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “host cell” includes an individual cell or cell culture that can be or has been a recipient of exogenous polynucleotide(s). Host cells include progeny of a single host cell, and the progeny may not necessarily be completely identical (in morphology or in genomic DNA complement) to the original parent cell due to natural, accidental, or deliberate mutation.

### Membrane-Bound Cytokines

As discussed above, there exists a need to provide ways to stimulate T cells to combat various acute and chronic diseases and to promulgate sufficient numbers of therapeutic T cells for adoptive immunotherapy. In particular, there remains a need for methods that circumvent the known challenges for using accessory cells, as accessory cells are relatively short-lived. As such, in a long-term culture system, these accessory cells must be continually obtained and replenished, which is problematic for treatment of immunodeficiencies in which accessory cells are affected.

As such, in one aspect, the present disclosure relates to a membrane-bound interleukin-7 (IL-7) construct, wherein the membrane-bound IL-7 construct comprises a signal sequence, an IL-7 amino acid sequence, a linker, and a membrane-insertion moiety. Moreover, in one embodiment, the present disclosure generally relates to a membrane bound construct comprising a first cytokine and a second cytokine, wherein the membrane-bound construct comprises a signal peptide; the first cytokine; a first linker; the second cytokine; a second linker; and a membrane-insertion moiety; wherein the first cytokine comprises IL-21 and the second cytokine comprises IL-7, or wherein the first cytokine comprises IL-7 and the second cytokine comprises IL-21. Furthermore, in one aspect, the present disclosure generally relates to a membrane bound construct comprising a first cytokine and a second cytokine, wherein the membrane-bound construct comprises a signal peptide; the first cytokine; a first linker; the second cytokine; a second linker; and a membrane-insertion moiety; wherein the first cytokine comprises IL-18 and the second cytokine comprises IL-21, or wherein the first cytokine comprises IL-21 and the second cytokine comprises IL-18.

In another embodiment, the present disclosure provides a chimeric membrane-bound interleukin-7 (IL-7) polypeptide comprising an IL-7 amino acid sequence, a linker, and a membrane-insertion moiety. In another embodiment, the present disclosure provides a membrane-bound combokine comprising a first cytokine, a linker, a second cytokine, and a membrane-insertion moiety. In another embodiment, the first cytokine comprises IL-21 and the second cytokine comprises IL-7, or the first cytokine comprises IL-7 and the second cytokine comprises IL-21. In another embodiment, the first cytokine comprises IL-18 and the second cytokine comprises IL-21, or the first cytokine comprises IL-21 and the second cytokine comprises IL-18.

As described further herein, the membrane-bound cytokines or combokines of the instant disclosure are capable of providing cytokine signals to enhance ex vivo expansion of T cells. In particular, the membrane-bound cytokines or combokines described herein provide, for example, IL-7, IL-21, and/or IL-18, to T cells without the need for the use of accessory cells, thereby providing a distinct advantage over accessory-cell based methods. Moreover, in some embodiments, use of the membrane-bound cytokines or combokines of the instant disclosure, including vectors encoding these membrane-bound cytokines or combokines, can reduce the cost of manufacturing as activation reagents can be linked with cytokines.

### Signal Peptide

In some embodiments, the extracellular domain of a polypeptide of the present disclosure can comprise a signal peptide or a leader that, for example, directs the nascent protein into the endoplasmic reticulum and subsequent translocation to the cell surface. It is understood that, in some embodiments, once a polypeptide containing a signal peptide is expressed at the cell surface, the signal peptide has generally been proteolytically removed during processing of the polypeptide (e.g., in the endoplasmic reticulum and translocation to the cell surface). Thus, in some embodiments, a polypeptide such as a membrane-bound polypeptide of the invention is generally expressed at a surface as a mature protein lacking the signal peptide, whereas the precursor form of the polypeptide includes the signal peptide. In other embodiments, the signal sequence or leader is a peptide sequence generally present at the N terminus of newly synthesized proteins that directs their entry into the secretory pathway. In other embodiments, the signal peptide is covalently joined to the N-terminus of the extracellular antigen-binding domain as a fusion protein.

In some embodiments, the membrane-bound polypeptides of the disclosure do not comprise a signal peptide.

In some embodiments, the membrane-bound polypeptides of the disclosure comprise a signal peptide. In one embodiment, the signal peptide comprises an amino acid sequence that is at least 75%, at least 80%, at least 85%, at least 90%, at least 95%, or at least 99% identical to the amino acid sequence of SEQ ID NO: 1. In another embodiment, the signal peptide comprises an amino acid sequence that is at least 90% or at least 95% identical to the amino acid sequence of SEQ ID NO: 1. In some embodiments, the signal peptide comprises the amino acid sequence of SEQ ID NO 1.

Any suitable signal peptide, as are well known in the art, can be applied to the constructs of the invention to provide surface expression. In some embodiments, the signal sequence is a human GM-CSF signal sequence. In other embodiments, the signal sequence is a signal sequence as provided in Huse, M., et al. Nature immunology vol. 7, 3 (2006): 247-55; Conradt, H. S., et al., Carbohydrate research vol. 149, 2 (1986): 443-50, e.g., an IL-2 signal sequence, e.g., an IL-3 signal sequence; or Goodwin, R. G., et al. Proceedings of the National Academy of Sciences of the United States of America vol. 86, 1 (1989): 302-6, e.g., an IL-7 signal sequence; the disclosure of each of which is hereby incorporated by reference in its entirety herein. In some embodiments, the signal sequence is an IL-2 signal sequence, an IL-3 signal sequence, an IL-7 signal sequence, or a human GM-CSF signal sequence.

### Cytokines

The chimeric membrane-bound polypeptides or nucleic acids of the present disclosure comprise or encode one or more cytokines. In some embodiments, a chimeric membrane-bound polypeptide or nucleic acid comprises or encodes at least one of IL-7, IL-21, and IL-18. In some embodiments, the chimeric membrane-bound polypeptide or nucleic acid comprises or encodes a first cytokine and a second cytokine. In some embodiments, the first cytokine comprises or is IL-7, IL-21, or IL-18. In some embodiments, the second cytokine comprises or is IL-7, IL-21, or IL-18.

The chimeric membrane-bound polypeptides of the present disclosure comprise one or more cytokines. In some embodiments, the membrane-bound polypeptide comprises at least one of IL-2, IL-4, IL-5, IL-7, IL-10, IL-12, IL-13, IL-15, IL-17, IL-18, IL-21, IL-35, and TGF-β. In some aspects, the construct comprises at least one of IL-1, IL-1α, IL-1β, IL-2, IL-3, IL-4, IL-5, IL-6, IL-7, IL-8, IL-9, IL-10, IL-11, IL-12, IL-12p40, IL-12p70, IL-13, IL-14, IL-15, IL-16, IL-17, IL-17A, IL-17B, IL-17C, IL-17D, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-36a, IL-36B, IL-367, IL-37, IL-38, IL-39, IL-40, IL-41, TNF-α, TNF-β, IFN-γ, IFN-α, IFN-β, TGF-β, GM-CSF, M-CSF, G-CSF, CCL2, CCL3, CCL4, CCL5, CCL11, CCL17, CCL19, CCL20, CXCL8, CXCL10, CXCL12, CXCL13, CXCL14, and CX3CL1. In some embodiments, a chimeric membrane-bound polypeptide comprises at least one of IL-7, IL-21, and IL-18.

In some embodiments, the membrane-bound polypeptide comprises a first cytokine and a second cytokine. In some aspects, the first cytokine comprises IL-2, IL-4, IL-5, IL-7, IL-10, IL-12, IL-13, IL-15, IL-17, IL-18, IL-21, IL-35, or TGF-β. In some aspects, the first cytokine comprises IL-1, IL-1a, IL-1B, IL-2, IL-3, IL-4, IL-5, IL-6, IL-7, IL-8, IL-9, IL-10, IL-11, IL-12, IL-12p40, IL-12p70, IL-13, IL-14, IL-15, IL-16, IL-17, IL-17A, IL-17B, IL-17C, IL-17D, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-36α, IL-36β, IL-36γ, IL-37, IL-38, IL-39, IL-40, IL-41, TNF-α, TNF-β, IFN-γ, IFN-α, IFN-β, TGF-β, GM-CSF, M-CSF, G-CSF, CCL2, CCL3, CCL4, CCL5, CCL11, CCL17, CCL19, CCL20, CXCL8, CXCL10, CXCL12, CXCL13, CXCL14, or CX3CL1. In some aspects, the second cytokine comprises IL-2, IL-4, IL-5, IL-7, IL-10, IL-12, IL-13, IL-15, IL-17, IL-18, IL-21, IL-35, or TGF-β. In some embodiments, the first cytokine is IL-7, IL-21, or IL-18.

In some embodiments, the second cytokine comprises IL-1, IL-1a, IL-1B, IL-2, IL-3, IL-4, IL-5, IL-6, IL-7, IL-8, IL-9, IL-10, IL-11, IL-12, IL-12p40, IL-12p70, IL-13, IL-14, IL-15, IL-16, IL-17, IL-17A, IL-17B, IL-17C, IL-17D, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-36α, IL-36β, IL-36γ, IL-37, IL-38, IL-39, IL-40, IL-41, TNF-α, TNF-β, IFN-γ, IFN-α, IFN-β, TGF-β, GM-CSF, M-CSF, G-CSF, CCL2, CCL3, CCL4, CCL5, CCL11, CCL17, CCL19, CCL20, CXCL8, CXCL10, CXCL12, CXCL13, CXCL14, or CX3CL1. In some embodiments, the second cytokine comprises or is IL-7, IL-21, or IL-18.

In some embodiments, the membrane-bound polypeptide comprises the amino acid sequence of an IL-7 isoform. In an embodiment, the membrane-bound polypeptide comprises a mature human IL-7 variant 1 long isoform. In another embodiment, the membrane-bound polypeptide IL-7 variant lacks exon 5 (IL-7Δ5). In another embodiment, the membrane-bound polypeptide IL-7 variant lacks exon 4 (IL-7Δ4). In another embodiment, the membrane-bound polypeptide IL-7 variant lacks exons 3 and 4 (IL-7Δ3,4). In another embodiment, the membrane-bound polypeptide IL-7 variant lacks exons 4 and 5 (IL-7Δ4,5). In some embodiments, the IL-7 comprises an amino acid sequence that is at least 75%, at least 80%, at least 85%, at least 90%, at least 95%, at least 96%, at least 97%, at least 98%, or at least 99% identical to the amino acid sequence of SEQ ID NO: 2. In some embodiments, the IL-7 comprises an amino acid sequence that is at least 90 or at least 95% identical to the amino acid sequence of SEQ ID NO: 2. In some embodiments, the IL-7 comprises the amino acid sequence of SEQ ID NO: 2.

In some embodiments, the membrane-bound polypeptide comprises the amino acid sequence of an IL-21 isoform. In an embodiment, the IL-21 isoform is the canonical IL-21 (full-length isoform). In another embodiment, the IL-21 isoform is a splice variant, such as IL-21 isoform 2 lacking exon 3 (IL-2143). In some embodiments, the IL-21 isoform comprises an amino acid sequence that is at least 75%, at least 80%, at least 85%, at least 90%, at least 95%, at least 96%, at least 97%, at least 98%, or at least 99% identical to the amino acid sequence of SEQ ID NO: 17. In some embodiments, the IL-21 isoform comprises an amino acid sequence that is at least 90 or at least 95% identical to the amino acid sequence of SEQ ID NO: 17. In some embodiments, the IL-21 isoform comprises the amino acid sequence of SEQ ID NO: 17. In some embodiments, the IL-21 isoform comprises an S108P substitution. In some embodiments, the membrane-bound polypeptide comprises or encodes a mature human IL-21 variant 1 isoform. In some embodiments, the IL-21 sequence is a human IL-21 sequence. Human IL-21 amino acid sequences include, for instance, but are not limited to, Genbank Accession Nos: AAU88182.1, EAX05226.1, CAI94500.1, CAJ47524.1, CAL81203.1, CAN87399.1, CAS03522.1, CAV33288.1, CBE74752.1, CBI70418.1, CBI85469.1, CBI85472.1, CBL93962.1, CCA63962.1, AAG29348.1, AAH66258.1, AAH66259.1, AAH66260.1, AAH66261.1, AAH66262.1, AAH69124.1, and ABG36529.1. Other human IL-21 sequences, as well as other IL-21 species can be employed in accordance with the disclosure. In some embodiments, the IL-21 amino acid sequence is the amino acid sequence of mature, human IL-21.

In some embodiments, the membrane-bound polypeptide comprises the amino acid sequence of an IL-18 isoform. In some embodiments, the IL-18 isoform comprises an amino acid sequence that is at least 75%, at least 80%, at least 85%, at least 90%, at least 95%, at least 96%, at least 97%, at least 98%, or at least 99% identical to the amino acid sequence of SEQ ID NO: 18. In some embodiments, the IL-18 isoform comprises an amino acid sequence that is at least 90 or at least 95% identical to the amino acid sequence of SEQ ID NO: 18. In some embodiments, the IL-18 isoform comprises the amino acid sequence of SEQ ID NO: 18. In some embodiments, the membrane-bound polypeptide comprises a human IL-18 isoform.

### Linkers

The chimeric membrane bound polypeptide of the present disclosure comprises at least one linker. In some embodiments, the construct comprises a first linker and a second linker. In certain embodiments, either or both of the first linker and the second linker can be any of the linkers described herein. In other embodiments, either or both of the first and second linkers comprises a GS (Gly-Ser) linker. In some embodiments, the second linker comprises the amino acid sequence of SEQ ID NO: 19.

In some embodiments, the linker is between the cytokine and the membrane-insertion moiety. In some embodiments, the linker is between a first cytokine and a second cytokine. In some embodiments, the linker is a GS (Gly-Ser) linker. As used herein, the term “GS linker” is used with reference to a linker that is comprised of and/or essentially consists of glycine and serine residues. In certain embodiments, the GS linker comprises the amino acid sequence of SEQ ID NO: 19.

In some embodiments, the linker is comprised of several immunoglobulin (Ig) hinge region (e.g., IgG4 hinge) and/or Ig constant domain regions (e.g., IgG CH1, CH2, CH3). Thus, in some embodiments, the linker comprises an IgG4 hinge region exemplified by the amino acid sequence of SEQ ID NO: 3. In some embodiments, the linker comprises an IgG4 CH2 domain exemplified by the amino acid sequence of SEQ ID NO: 4. In some embodiments, the linker comprises an IgG4 CH3 domain exemplified by the amino acid sequence of SEQ ID NO: 5. In some embodiments, the linker is a chimeric linker comprising the amino acid sequence of SEQ ID NO: 20.

In some embodiments, the linker comprises an amino acid sequence that is at least 75%, at least 80%, at least 85%, at least 90%, at least 95%, or at least 99% identical to the amino acid sequence of SEQ ID NO: 3; an amino acid sequence that is at least 75%, at least 80%, at least 85%, at least 90%, at least 95%, at least 96%, at least 97%, at least 98%, or at least 99% identical to the amino acid sequence of SEQ ID NO: 4, and/or an amino acid sequence that is at least 75%, at least 80%, at least 85%, at least 90%, at least 95%, at least 96%, at least 97%, at least 98%, or at least 99% identical to the amino acid sequence of SEQ ID NO: 5. In some embodiments, the linker comprises an amino acid sequence that is at least 90 or at least 95% identical to the amino acid sequence of SEQ ID NO: 3, an amino acid sequence that is at least 90 or at least 95% identical to the amino acid sequence of SEQ ID NO: 4, and/or an amino acid sequence that is at least 90 or at least 95% identical to the amino acid sequence of SEQ ID NO: 5. In some embodiments, the linker comprises the amino acid sequence of SEQ ID NO: 3, the amino acid sequence of SEQ ID NO: 4, and/or the amino acid sequence of SEQ ID NO: 5.

In some embodiments, a chimeric linker comprises, from amino terminus to carboxy terminus, an amino acid sequence that is at least 90%, at least 95%, at least 96%, at least 97%, at least 98%, or at least 99% identical to the amino acid sequence of SEQ ID NO: 3; an amino acid sequence that is at least at least 90%, at least 95%, at least 96%, at least 97%, at least 98%, or at least 99% identical to the amino acid sequence of SEQ ID NO: 4, and an amino acid sequence that is at least 90%, at least 95%, at least 96%, at least 97%, at least 98%, or at least 99% identical to the amino acid sequence of SEQ ID NO: 5. In one embodiment, the linker comprises, from amino terminus to carboxy terminus, an amino acid sequence comprising the amino acid sequence of SEQ ID NO: 3, an amino acid comprising the amino acid sequence of SEQ ID NO: 4, and an amino acid sequence comprising the amino acid sequence of SEQ ID NO: 5. In another embodiment, the chimeric linker comprises an amino acid sequence that is at least 90%, at least 95%, at least 96%, at least 97%, at least 98%, or at least 99% identical to the amino acid sequence of SEQ ID NO: 20. In another embodiment, the chimeric linker comprises the amino acid sequence of SEQ ID NO: 20.

### Membrane-Insertion Moieties

The chimeric membrane-bound polypeptide comprises a membrane-insertion moiety. In some embodiments, the membrane insertion moiety comprises a transmembrane domain. In some embodiments, the transmembrane domain comprises a human transmembrane domain. In some embodiments, the transmembrane domain is derived either from a natural or from a synthetic source. In some embodiments, where the source is natural, the domain is derived from any membrane-bound or transmembrane protein, e.g., a Type I transmembrane protein. In some embodiments, where the source is synthetic, the transmembrane domain is any artificial sequence that facilitates insertion into a cell membrane, e.g., an artificial hydrophobic sequence.

Examples of the transmembrane regions of particular use in this disclosure include, without limitation, transmembrane domains derived from (i.e. comprise at least the transmembrane region(s) of) the alpha, beta or zeta chain of the T-cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In some instances, a triplet of phenylalanine, tryptophan and valine is found at each end of a synthetic transmembrane domain.

In some embodiments, the membrane insertion moiety is selected from the group consisting of a CD4 transmembrane domain, a CD8 transmembrane domain, and a CD28 transmembrane domain. In some embodiments, the membrane insertion moiety comprises or is a human CD4 transmembrane domain. In some embodiments, the human CD4 transmembrane domain comprises an amino acid sequence that is at least 95%, at least 96%, at least 97%, at least 98%, or at least 99% identical to SEQ ID NO: 6. In some embodiments, the membrane-insertion moiety comprises an amino acid sequence that is at least 95% identical to SEQ ID NO: 6. In some embodiments, the membrane-insertion moiety comprises the amino acid sequence of SEQ ID NO: 6.

In some embodiments, the membrane insertion moiety comprises a GPI-anchoring peptide. Engineering a protein of interest for linkage to GPI constitutes a post-translational modification in which GPI embeds into the lipid membrane thereby targeting the protein for membrane insertion. The cellular process for the attachment of GPI to proteins in vivo is well known and well-characterized. GPI-anchoring is a post-translational modification in which the GPI is assembled on the endoplasmic reticulum (ER) membrane and is transferred to the target protein immediately after its translocation into the ER lumen, with concomitant cleavage of a carboxy-terminal GPI-anchor sequence by a transamidase enzyme. Lipid remodeling and/or carbohydrate side-chain modifications may then occur in the ER and after transport to the Golgi apparatus.

C-terminal GPI anchor sequences for membrane targeting are known in the art. Hundreds of GPI-anchored proteins have been identified in many eukaryotes, ranging from protozoa and fungi to humans. Any suitable GPI anchor sequence may be used according to the invention. Examples of mammalian proteins having known C-terminal GPI anchor sequences include CD59, CD52, CD55 (DAF), CD87 (uPAR), Neural cell adhesion molecule 120 (NCAM-120), Neural cell adhesion molecule TAG-1, CD58, Fcylll receptor, Ciliary neurotrophic factor receptor (CNTFR) a subunit, Glial-cell-derived neurotrophic factor receptor (GDNFR) a subunit, CD14, and the Glypican family of GPI-anchored proteoglycans. Exemplary GPI-anchoring sequences are disclosed in US20240415976 A1, the disclosure of which is expressly incorporated by reference in its entirety.

### Single and Multiple Cytokine Polypeptides

In one aspect, the present disclosure generally relates to a membrane-bound Interleukin-7 (IL-7) polypeptide comprising comprises an IL-7 amino acid sequence, a linker, and a membrane-insertion moiety as described herein. In some embodiments, the membrane-bound IL-7 polypeptide comprises an amino acid sequence at least 95%, at least 96%, at least 97%, at least 98%, or at least 99% identical to the amino acid sequence of SEQ ID NO: 7. In some embodiments, the membrane-bound IL-7 polypeptide is encoded by a nucleotide sequence at least 70%, at least 75%, at least 80%, at least 85%, at least 90%, at least 95%, at least 96%, at least 97%, at least 98%, or at least 99% identical to SEQ ID NO: 8. In some embodiments, the membrane-bound IL-7 polypeptide comprises an amino acid sequence at least at least 90 or at least 95% identical to the amino acid sequence of SEQ ID NO: 7 or is encoded by a nucleic acid sequence at least 90 or at least 95% identical to the nucleotide sequence of SEQ ID NO: 8. In some embodiments, the membrane-bound IL-7 polypeptide comprises the amino acid sequence of SEQ ID NO: 7 or is encoded by the nucleotide sequence of SEQ ID NO: 8. In some embodiments, the membrane-bound polypeptide is capable of activating STAT3 and/or STAT5 signaling in a cell. In some embodiments, the cell is a T-cell.

In another aspect, the membrane-bound polypeptide is a combokine further comprising a second cytokine in addition to IL-7. In one embodiment, the second cytokine is IL-21. In some embodiments, IL-21 comprises an S108P substitution. In some embodiments, the combokine is capable of activating STAT3 and/or STAT5 signaling in a cell. In some embodiments, the cell is a T-cell.

In another embodiment, the present disclosure provides a membrane bound construct, e.g., a dual membrane bound cytokine construct, comprising a first cytokine and a second cytokine, wherein the construct comprises a signal peptide; the first cytokine; a first linker; the second cytokine; a second linker; and a membrane-insertion moiety; wherein the first cytokine comprises IL-21 and the second cytokine comprises IL-7, or wherein the first cytokine comprises IL-7 and the second cytokine comprises IL-21. In some embodiments, the signal sequence, the IL-7 amino acid sequence, the IL-21 amino acid sequence, the first linker, the second linker, and the membrane-insertion moiety can be any of those described herein. In some aspects, the first cytokine comprises IL-21 and the second cytokine comprises IL-7.

In another embodiment, an IL-21/IL-7 combokine comprises a first cytokine, a linker, a second cytokine, and a membrane-insertion moiety, wherein the first cytokine comprises IL-21 and the second cytokine comprises IL-7. In some embodiments, the IL-21/IL-7 combokine comprises an amino acid sequence at least 70%, at least 75%, at least 80%, at least 85%, at least 90%, at least 95%, at least 96%, at least 97%, at least 98%, or at least 99% identical to SEQ ID NO: 9. In other embodiments, the IL-21/IL-7 combokine is encoded by a nucleotide sequence at least 70%, at least 75%, at least 80%, at least 85%, at least 90%, at least 95%, at least 96%, at least 97%, at least 98%, or at least 99% identical to SEQ ID NO: 13. In some embodiments, the IL-21/IL-7 combokine comprises an amino acid sequence at least 90 or at least 95% identical to SEQ ID NO: 9 or is encoded by a nucleotide sequence at least 90 or at least 95% identical to SEQ ID NO: 13. In some embodiments, the IL-21/IL-7 combokine comprises the amino acid sequence of SEQ ID NO: 9 or is encoded by the nucleotide sequence of SEQ ID NO: 13.

In another embodiment, an IL-7/IL-21 combokine comprises a first cytokine, a linker, a second cytokine, and a membrane-insertion moiety, where the first cytokine comprises IL-7 and the second cytokine comprises IL-21. In some embodiments, the membrane-bound polypeptide comprises an amino acid sequence at least 70%, at least 75%, at least 80%, at least 85%, at least 90%, at least 95%, at least 96%, at least 97%, at least 98%, or at least 99% identical to SEQ ID NO: 10. In other embodiments, the IL-7/IL-21 combokine is encoded by a nucleotide sequence at least 70%, at least 75%, at least 80%, at least 85%, at least 90%, at least 95%, at least 96%, at least 97%, at least 98%, or at least 99% identical to SEQ ID NO: 10. In some embodiments, the IL-7/IL-21 combokine comprises an amino acid sequence at least 90 or at least 95% identical to SEQ ID NO: 9 or is encoded by a nucleotide sequence at least 90 or at least 95% identical to SEQ ID NO: 14. In some embodiments, the IL-7/IL-21 combokine comprises the amino acid sequence of SEQ ID NO: 9 or is encoded by the nucleotide sequence of SEQ ID NO: 14.

In another aspect, the present disclosure provides a membrane bound construct, e.g., a dual membrane bound cytokine construct, comprising a first cytokine and a second cytokine, wherein the membrane-bound construct comprises a signal peptide; the first cytokine; a first linker; the second cytokine; a second linker; and a membrane-insertion moiety; wherein the first cytokine comprises IL-18 and the second cytokine comprises IL-21, or wherein the first cytokine comprises IL-21 and the second cytokine comprises IL-18. In some aspects, the signal sequence, the IL-21 amino acid sequence, the IL-18 amino acid sequence, the first linker, the second linker, and the membrane-insertion moiety can be any of those described herein.

In another embodiment, the membrane-bound polypeptide is a combokine comprising a first cytokine, a linker, a second cytokine, and a membrane-insertion moiety, wherein the first cytokine comprises IL-18 and the second cytokine comprises IL-21 (an “IL-18/IL-21 combokine”), or wherein the first cytokine comprises IL-21 and the second cytokine comprises IL-18 (an “IL-21/IL-18 combokine”). The IL-21 amino acid sequence, the IL-18 amino acid sequence, the linker, and the membrane-insertion moiety can be any of those described herein.

In one embodiment, an IL-18/IL-21 combokine comprises a first cytokine, a linker, a second cytokine, and a membrane-insertion moiety, wherein the first cytokine comprises IL-18 and the second cytokine comprises IL-21. In some embodiments, the IL-18/IL-21 combokine comprises an amino acid sequence at least 70%, at least 75%, at least 80%, at least 85%, at least 90%, at least 95%, at least 96%, at least 97%, at least 98%, or at least 99% identical to SEQ ID NO: 11. In other embodiments, the IL-18/IL-21 combokine is encoded by a nucleotide sequence at least 70%, at least 75%, at least 80%, at least 85%, at least 90%, at least 95%, at least 96%, at least 97%, at least 98%, or at least 99% identical to SEQ ID NO: 15. In some embodiments, the IL-18/IL-21 combokine comprises an amino acid sequence at least 90 or at least 95% identical to SEQ ID NO: 11 or is encoded by a nucleotide sequence at least 90 or at least 95% identical to SEQ ID NO: 15. In some embodiments, the IL-18/IL-21 combokine comprises the amino acid sequence of SEQ ID NO: 11 or is encoded by the nucleotide sequence of SEQ ID NO: 15.

In another embodiment, an IL-21/IL-18 combokine comprises a first cytokine, a linker, a second cytokine, and a membrane-insertion moiety, where the first cytokine comprises IL-21 and the second cytokine comprises IL-18. In some embodiments, the membrane-bound polypeptide comprises an amino acid sequence at least 70%, at least 75%, at least 80%, at least 85%, at least 90%, at least 95%, at least 96%, at least 97%, at least 98%, or at least 99% identical to SEQ ID NO: 12. In other embodiments, the IL-21/IL-18 combokine is encoded by a nucleotide sequence at least 70%, at least 75%, at least 80%, at least 85%, at least 90%, at least 95%, at least 96%, at least 97%, at least 98%, or at least 99% identical to SEQ ID NO: 16. In some embodiments, the IL-21/IL-18 combokine comprises an amino acid sequence at least 90 or at least 95% identical to SEQ ID NO: 12 or is encoded by a nucleotide sequence at least 90 or at least 95% identical to SEQ ID NO: 16. In some embodiments, the IL-21/IL-18 combokine comprises the amino acid sequence of SEQ ID NO: 12 or is encoded by the nucleotide sequence of SEQ ID NO: 16.

Amino acid and nucleotide sequences corresponding to the membrane-bound cytokines or combokines are shown in Table 1.

### Polynucleotides and Vectors

In another aspect, the present invention provides a polynucleotide encoding a membrane-bound cytokine or combokine. In some embodiments, the polynucleotide is a recombinant DNA molecule. In some embodiments, the polynucleotide is a recombinant RNA molecule. In some embodiments, a vector comprises the nucleic acid. In some embodiments, the vector is an expression vector.

In an embodiment, the polynucleotide or vector comprises a signal sequence encoding a signal peptide at the N-terminal end of the chimeric cytokine or combokine, which is cleaved from the protein (e.g., antigen binding domain) during cellular processing and localization of the chimeric cytokine or combokine to the cellular membrane or secretion of a protein therefrom.

In some embodiments, the signal sequence is a signal sequence as provided in Huse, M., et al. Nature immunology vol. 7, 3 (2006): 247-55; Conradt, H. S., et al., Carbohydrate research vol. 149, 2 (1986): 443-50, e.g., an IL-2 signal sequence, e.g., an IL-3 signal sequence; or Goodwin, R. G., et al. Proceedings of the National Academy of Sciences of the United States of America vol. 86, 1 (1989): 302-6, e.g., an IL-7 signal sequence; the disclosure of each of which is hereby incorporated by reference in its entirety herein. In some embodiments, the signal sequence is an IL-2 signal sequence, an IL-3 signal sequence, an IL-7 signal sequence, a human GM-CSF signal sequence, or a CD8 signal sequence.

In some embodiments, the signal sequence is a human GM-CSF signal sequence. In some embodiments, the human GM-CSF signal sequence comprises an amino acid sequence that is at least 95%, at least 96%, at least 97%, at least 98%, or at least 99% identical to the amino acid sequence of SEQ ID NO: 1. In some embodiments, the human GM-CSF signal sequence comprises the amino acid sequence of SEQ ID NO: 1.

In some embodiments, the polynucleotide encoding the membrane-bound cytokine or combokine is operably linked to a transcriptional control element, e.g., a promoter, and enhancer, and so forth. Suitable promoter and enhancer elements are known to those of skill in the art. In some embodiments, the polynucleotide encoding the membrane-bound cytokine or combokine is operably linked to a promoter. Suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, and so forth, is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), and so forth), tetracycline regulated promoters, (e.g., promoter systems including TetActivators, TetON, TetOFF, and so forth), steroid regulated promoters (e.g., rat glucocorticoid receptor promoter systems, human estrogen receptor promoter systems, retinoid promoter systems, thyroid promoter systems, ecdysone promoter systems, mifepristone promoter systems, and so forth), metal regulated promoters (e.g., metallothionein promoter systems, and so forth), pathogenesis-related regulated promoters (e.g., salicylic acid regulated promoters, ethylene regulated promoters, benzothiadiazole regulated promoters, and so forth), temperature regulated promoters (e.g., heat shock inducible promoters (e.g., HSP-70, HSP-90, soybean heat shock promoter, and so forth), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GALI promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, the polynucleotide or vector contains a promoter that is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, and so forth known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

A polynucleotide encoding the membrane-bound cytokine or combokine of the present disclosure can be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pST-Ki, pST-KiT, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863; Jomary et al., Gene Ther. (1997) 4:683 690, Rolling et al., Hum. Gene Ther. (1999) 10:641-648; Ali et al., Hum. Mol. Genet. (1996) 5:591-594; Srivastava in WO 93/09239, Samulski et al., J. Vir. (1989) 63:3822-3828; Mendelson et al., Virol. (1988) 166:154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector can be introduced into a cell to express the chimeric membrane-bound polypeptide. Accordingly, an expression vector (e.g., a lentiviral vector) of the present invention may comprise a nucleic acid encoding the membrane-bound cytokine or combokine of the present disclosure. In some embodiments, the expression vector will comprise additional elements that will aid in the functional expression of the membrane-bound cytokine or combokine. In some embodiments, an expression vector comprising a nucleic acid encoding the membrane-bound cytokine or combokine further comprises a mammalian promoter. In one aspect, the vector further comprises an elongation-factor-1-alpha promoter (EF-1a promoter). Use of an EF-1a promoter may increase the efficiency in expression of downstream transgenes. Physiologic promoters (e.g., an EF-1a promoter) may be less likely to induce integration mediated genotoxicity, and may abrogate the ability of the retroviral vector to transform stem cells. Other physiological promoters suitable for use in a vector are known to those of skill in the art and may be incorporated into a vector of the present invention. In some embodiments, the vector further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one aspect, a vector of the present invention includes a 3′ U3 deleted LTR. Accordingly, a vector of the present invention may comprise any combination of the elements described herein to enhance the efficiency of functional expression of transgenes.

In some embodiments, a polynucleotide encoding the membrane-bound cytokine or combokine is RNA, e.g., in vitro synthesized RNA. Methods for in vitro synthesis of RNA are known to those of skill in the art; any known method can be used to synthesize RNA comprising a sequence encoding the membrane-bound cytokine or combokine of the present disclosure. Methods for introducing RNA into a host cell are known in the art. See, e.g., Zhao et al. Cancer Res. (2010) 15:9053. Introducing RNA comprising a nucleotide sequence encoding the membrane-bound cytokine or combokine of the present disclosure into a host cell can be carried out in vitro, ex vivo or in vivo. For example, a host cell can be electroporated in vitro or ex vivo with RNA comprising a nucleotide sequence encoding the membrane-bound cytokine or combokine of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82).

In one aspect, the present disclosure generally relates to an isolated polynucleotide comprising a nucleotide sequence encoding the membrane-bound cytokine or combokine as described herein. In one aspect, the present disclosure generally relates to a vector comprising a polynucleotide as described herein. Furthermore, in one aspect, the present disclosure generally relates to an isolated host cell comprising a membrane-bound cytokine or combokine as described herein and/or a polynucleotide as described herein and/or a vector as described herein.

### Membrane Vesicles

In one aspect, the present disclosure provides a membrane vesicle comprising a membrane-bound cytokine or combokine and/or a nucleic acid encoding a membrane-bound cytokine or combokine as described herein. As used herein, the term “membrane vesicle” (or “MV”) is used interchangeably with reference to a lipid-bilayer encapsulated extracellular vesicle (EV), which may be secreted from a cell, such as an exosome. MVs or EVs play crucial roles in intercellular communication and can transport various biological molecules, including proteins, lipids, and nucleic acids. MVs or EVs encompass a wide range of sizes, typically categorized into two main groups: small EVs (sEVs) and large EVs (IEVs). Small EVs, which include exosomes, generally range from 30 to 150 nanometers in diameter. These vesicles originate from the endosomal system and are released when multivesicular bodies fuse with the plasma membrane. Large EVs, on the other hand, are typically larger than 200 nanometers and can reach sizes up to several micrometers. This category includes microvesicles, which bud directly from the plasma membrane, and apoptotic bodies. While the endosomal mechanism of sEV formation is widely accepted, some evidence suggests that sEVs can also form directly from the plasma membrane, or from intracellular compartments connected via a channel to the cell surface. EVs in this application include both sEVs and IEVs.

In some embodiments, the membrane vesicles are generated as described in Oyer, J. L., et al., Biology of blood and marrow transplantation: Journal of the American Society for Blood and Marrow Transplantation vol. 21, 4 (2015), which is hereby incorporated by reference in its entirety herein. In some embodiments, membrane vesicle fragments are purified by a sucrose gradient and tangential flow filtration (TFF). In some embodiments, the membrane vesicles are generated by the disruption of cells comprising a membrane-bound cytokine or combokine as described herein. A number of methods of cell disruption are well-known in the art including, but not limited to, mechanical homogenization such as with a handheld or motorized device, ultrasonic homogenization, pressure homogenization, temperature treatments comprising repeated cycles of freezing and thawing, osmotic lysis in hypotonic solutions, chemical lysis including the use of various detergents or solvents, nitrogen cavitation, among others. The skilled artisan would recognize which method of disruption would be most applicable to specific applications of generating membrane vesicles. In some embodiments, the method of disruption is nitrogen cavitation. This method involves placing a culture cells in a pressure vessel, often called a “bomb”, and equilibrating the cells at high pressure under a nitrogen-rich atmosphere, thereby resulting in increased nitrogen and other gasses dissolved in the cytoplasm of the cells. The sudden exposure of the cells to atmospheric pressure results in nitrogen bubbles forming in the cytoplasm of the cells, which results in cell lysis. Pressure and release time can be varied to result in differing degrees of cell lysis and preservation of cellular, membrane, protein, and organelle structures. For example, relatively low pressures will disrupt the plasma membrane and endoplasmic reticulum only, while high pressures will result in the disruption of the nucleus and other organelles including lysosomes and mitochondria. In some embodiments, the pressure vessel, often called a “bomb”, consists of a thick stainless steel or similar metal casing that is capable of withstanding high pressure, with an inlet for delivery of nitrogen gas from a tank and an outlet port with an adjustable discharge valve. The skilled artisan would recognize the precise conditions of nitrogen cavitation sufficient to produce the membrane vesicles of the invention and their use in the methods of the invention.

### Pharmaceutical Compositions

In some embodiments, the present disclosure encompasses the preparation and use of pharmaceutical compositions comprising a membrane-bound cytokine or combokine as described herein in combination with one or more pharmaceutically acceptable carriers. In some embodiments, the pharmaceutical composition comprises a cell, vesicle, or vector comprising a membrane-bound cytokine or combokine and/or a nucleic acid encoding a membrane-bound cytokine or combokine as described herein. In some embodiments, the composition comprises mixtures of membrane vesicles comprising more than one membrane-bound cytokine or combokine as described herein. Such a pharmaceutical compositions may consist of the active ingredient alone (e.g., a cell or vector preparation comprising and/or encoding a membrane-bound cytokine or combokine), or in combination with at least one additional active ingredient at effective doses in form(s) suitable for administration to a subject for activating and expanding T cells from a subject.

As used herein, the term “pharmaceutically acceptable carrier” means a chemical composition with which the active ingredient may be combined and which, following the combination, can be used to administer the active ingredient to a subject or activate and expand T cells from a subject. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates (e.g. monkeys, e.g. Macaca fascicularis, Macaca mulatta), cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the invention may be prepared, packaged, or sold in formulations suitable for oral, rectal, vaginal, parenteral, topical, pulmonary, intranasal, intra-lesional, buccal, ophthalmic, intravenous, intra-organ or another route of administration or use. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or for use in the activation and expansion of T cells from a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

In some embodiments, cells (e.g., T cells, NK cells) comprising nucleic acids or vectors encoding the membrane-bound cytokines or combokines of the disclosure or vectors encoding the membrane-bound cytokines of the disclosure can be administered to an animal, preferably a human. In some embodiments, prior to use, the cells comprising the foregoing nucleic acids or vectors are expanded such that the amount of cells administered can range from about 1 million cells to about 300 billion. In some embodiments, where membrane vesicles comprising the membrane-bound cytokines or combokines of the disclosure are themselves are administered, either with or without T cells expanded thereby, they can be administered in an amount ranging from about 100,000 to about ten billion cells or vesicles wherein the cells and vesicles are infused into the animal, preferably, a human patient in need thereof. In some embodiments, the precise dosage administered varies depending upon any number of factors, including but not limited to, the type of animal and type of disease state being treated, the age of the animal and the route of administration.

In some embodiments, the cells or vectors of the present disclosure are administered to an animal as frequently as several times daily, or they may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

In some embodiments, a cell, vector, or membrane vesicle comprising or encoding the membrane-bound cytokines or combokines is co-administered with one or more other therapeutically active compounds (cytokines, chemotherapeutic and/or antiviral drugs, among many others). In some embodiments, these compound(s) are administered an hour, a day, a week, a month, or even more, in advance of the cell, vector, or membrane vesicle comprising or encoding the membrane-bound cytokines or combokines, or any permutation thereof. Further, the compound(s) may be administered an hour, a day, a week, or even more, after administration of the cell, vector, or membrane vesicle comprising or encoding the membrane-bound cytokines or combokines, or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds, the vector expressing the membrane-bound polypeptide, the membrane vesicle comprising the membrane-bound polypeptide (or cells expanded thereby), and the like.

### Methods of Use

In one aspect, the present invention provides a method for stimulating and expanding a T cell (e.g., a cytotoxic T cell, a regulatory T cell (Treg), CAR-T cell etc.). In one embodiment, the method comprises contacting the T cell with a nucleic acid (e.g., expression vector) encoding a membrane-bound polypeptide described herein, a membrane vesicle comprising a membrane-bound polypeptide described herein, or a pharmaceutical composition as described herein.

In some embodiments, the T cell is an autologous T cell. In some embodiments, the T cell is a human T cell. In some embodiments, the cell is a cytotoxic T cell. In some embodiments, the T cell is a CAR-T cell. In some embodiments, the T cell is a T cell of a subset of interest. One skilled in the art would understand, based upon the disclosure provided herein, that T cell subsets include T helper (T_{H1} and T_{H2}) CD4 expressing, cytotoxic T lymphocyte (CTL) (Tc1 or Tc2) T regulatory (TREG), T_{C/S}, naïve, memory, central memory, effector memory, and γδT cells. In some aspects, the T cell is used in a method of treating cancer.

In another aspect, the present disclosure provides a method for stimulating and expanding a cytotoxic T cell or CAR-T cell, comprising contacting the cytotoxic T cell or CAR-T cell with a nucleic acid (e.g., expression vector) encoding a membrane-bound polypeptide described herein, a membrane vesicle composition comprising a membrane-bound polypeptide described herein, or a pharmaceutical composition as described herein. In certain embodiments, the membrane vesicle compositions offer several advantages over co-culture with aAPC cells including, but not limited to, obviating the need for inactivation of the aAPCs through treatments such as cytotoxic chemicals, irradiation, or the like that may negatively affect the cultured T cells. In some embodiments, the cytotoxic T cell is a human T cell. In some embodiments, the cytotoxic T cell is a non-human primate T cell. In some embodiments, the non-human primate is Macaca fascicularis. In some embodiments, the non-human primate is Macaca mulatta. In some embodiments, the cytotoxic T cells or CAR-T cells, such as those expanded stimulated and/or expanded using the methods described herein, are used in a method of treating cancer.

TABLE 1
Amino acid and nucleotide sequences corresponding to the membrane-bound
cytokines or combokines disclosed herein.

| SEQ ID | | |
|---|---|---|
| NO | Description | Sequence |
| 1 | Human GM-CSF Signal | MWLQSLLLLGTVACSIS |
| | Peptide | |
| 2 | Mature human IL-7 variant 1 | DCDIEGKDGKQYESVLMVSIDQLLDSMKEIGSNCL |
| | long isoform | NNEFNFFKRHICDANKEGMFLFRAARKLRQFLKMN |
| | | STGDFDLHLLKVSEGTTILLNCTGQVKGRKPAALG |
| | | EAQPTKSLEENKSLKEQKKLNDLCFLKRLLQEIKT |
| | | CWNKILMGTKEH |
| 3 | IgG4 hinge region | ESKYGPPCPPCP |
| 4 | IgG4 CH2 domain | APEFLGGPSVFLFPPKPKDTLMISRTPEVTCVVVD |
| | | VSQEDPEVQFNWYVDGVEVHNAKTKPREEQFNSTY |
| | | RVVRVLTVLHQDWLNGKEYKCKVSNKGLPSSIEKT |
| | | ISKAK |
| 5 | IgG4 CH3 domain | GQPREPQVYTLPPSQEEMTKNQVSLTCLVKGFYPS |
| | | DIAVEWESNGQPEDNYKTTPPVLDSDGSFFLYSRL |
| | | TVDKSRWQEGNVFSCSVMHEALHNHYTQKSLSLSP |
| | | GK |
| 6 | Hu CD4 transmembrane | MALIVLGGVAGLLLFIGLGIFF |
| | domain | |
| 7 | Membrane-bound IL-7 | MWLQSLLLLGTVACSISDCDIEGKDGKQYESVLMV |
| | (mIL-7) | SIDQLLDSMKEIGSNCLNNEFNFFKRHICDANKEG |
| | | MFLFRAARKLRQFLKMNSTGDFDLHLLKVSEGTTI |
| | | LLNCTGQVKGRKPAALGEAQPTKSLEENKSLKEQK |
| | | KLNDLCFLKRLLQEIKTCWNKILMGTKEHESKYGP |
| | | PCPPCPAPEFLGGPSVFLFPPKPKDTLMISRTPEV |
| | | TCVVVDVSQEDPEVQFNWYVDGVEVHNAKTKPREE |
| | | QFNSTYRVVRVLTVLHQDWLNGKEYKCKVSNKGLP |
| | | SSIEKTISKAKGQPREPQVYTLPPSQEEMTKNQVS |
| | | LTCLVKGFYPSDIAVEWESNGQPEDNYKTTPPVLD |
| | | SDGSFFLYSRLTVDKSRWQEGNVFSCSVMHEALHN |
| | | HYTQKSLSLSPGKMALIVLGGVAGLLLFIGLGIFF |
| 8 | Human IL-7-codon | GATTGCGATATCGAAGGCAAAGATGGTAAGCAGTA |
| | optimized | CGAAAGTGTTCTGATGGTTTCCATTGACCAACTGC |
| | | TTGACTCCATGAAGGAAATTGGTTCTAATTGTCTT |
| | | AATAATGAGTTTAACTTTTTTAAGAGACACATTTG |
| | | CGATGCCAATAAGGAGGGAATGTTCCTTTTCCGCG |
| | | CCGCAAGAAAGCTGAGGCAATTCTTGAAGATGAAC |
| | | TCCACCGGCGATTTCGACTTGCACCTTTTGAAGGT |
| | | CAGCGAAGGAACGACCATTTTGCTGAACTGCACCG |
| | | GCCAGGTGAAGGGTCGGAAGCCAGCGGCACTGGGA |
| | | GAGGCGCAGCCGACTAAGAGCCTTGAGGAAAATAA |
| | | GTCTCTTAAAGAACAGAAAAAACTTAACGACCTGT |
| | | GTTTTCTGAAACGGTTGCTTCAGGAAATCAAGACC |
| | | TGTTGGAACAAAATACTGATGGGGACTAAAGAGCA |
| | | T |
| 9 | Membrane-bound IL-21GS7 | MWLQSLLLLGTVACSISHKSSSQGQDRHMIRMRQL |
| | amino acid sequence | IDIVDQLKNYVNDLVPEFLPAPEDVETNCEWSAFS |
| | | CFQKAQLKSANTGNNERIINVSIKKLKRKPPSTNA |
| | | GRRQKHRLTCPSCDSYEKKPPKEFLERFKSLLQKM |
| | | IHQHLSSRTHGSEDSGGGGSGGGGSGGGGSDCDIE |
| | | GKDGKQYESVLMVSIDQLLDSMKEIGSNCLNNEFN |
| | | FFKRHICDANKEGMFLFRAARKLRQFLKMNSTGDF |
| | | DLHLLKVSEGTTILLNCTGQVKGRKPAALGEAQPT |
| | | KSLEENKSLKEQKKLNDLCFLKRLLQEIKTCWNKI |
| | | LMGTKEHESKYGPPCPPCPAPEFLGGPSVFLFPPK |
| | | PKDTLMISRTPEVTCVVVDVSQEDPEVQFNWYVDG |
| | | VEVHNAKTKPREEQFNSTYRVVSVLTVLHQDWLNG |
| | | KEYKCKVSNKGLPSSIEKTISKAKGQPREPQVYTL |
| | | PPSQEEMTKNQVSLTCLVKGFYPSDIAVEWESNGQ |
| | | PENNYKTTPPVLDSDGSFFLYSRLTVDKSRWQEGN |
| | | VFSCSVMHEALHNHYTQKSLSLSLGKMALIVLGGV |
| | | AGLLLFIGLGIFFCVRCRHRR |
| 10 | Membrane-bound IL-7GS21 | MWLQSLLLLGTVACSISDCDIEGKDGKQYESVLMV |
| | amino acid sequence | SIDQLLDSMKEIGSNCLNNEFNFFKRHICDANKEG |
| | | MFLFRAARKLRQFLKMNSTGDFDLHLLKVSEGTTI |
| | | LLNCTGQVKGRKPAALGEAQPTKSLEENKSLKEQK |
| | | KLNDLCFLKRLLQEIKTCWNKILMGTKEHGGGGSG |
| | | GGGSGGGGSHKSSSQGQDRHMIRMRQLIDIVDQLK |
| | | NYVNDLVPEFLPAPEDVETNCEWSAFSCFQKAQLK |
| | | SANTGNNERIINVSIKKLKRKPPSTNAGRRQKHRL |
| | | TCPSCDSYEKKPPKEFLERFKSLLQKMIHQHLSSR |
| | | THGSEDSESKYGPPCPPCPAPEFLGGPSVFLFPPK |
| | | PKDTLMISRTPEVTCVVVDVSQEDPEVQFNWYVDG |
| | | VEVHNAKTKPREEQFNSTYRVVSVLTVLHQDWLNG |
| | | KEYKCKVSNKGLPSSIEKTISKAKGQPREPQVYTL |
| | | PPSQEEMTKNQVSLTCLVKGFYPSDIAVEWESNGQ |
| | | PENNYKTTPPVLDSDGSFFLYSRLTVDKSRWQEGN |
| | | VFSCSVMHEALHNHYTQKSLSLSLGKMALIVLGGV |
| | | AGLLLFIGLGIFFCVRCRHRR |
| 11 | Membrane-bound IL-18GS21 | MWLQSLLLLGTVACSISYFGKLESKLSVIRNLNDQ |
| | amino acid sequence | VLFIDQGNRPLFEDMTDSDCRDNAPRTIFIISMYK |
| | | DSQPRGMAVTISVKCEKISTLSCENKIISFKEMNP |
| | | PDNIKDTKSDIIFFQRSVPGHDNKMQFESSSYEGY |
| | | FLACEKERDLFKLILKKEDELGDRSIMFTVQNEDG |
| | | GGGSGGGGSGGGGSHKSSSQGQDRHMIRMRQLIDI |
| | | VDQLKNYVNDLVPEFLPAPEDVETNCEWSAFSCFQ |
| | | KAQLKSANTGNNERIINVSIKKLKRKPPSTNAGRR |
| | | QKHRLTCPSCDSYEKKPPKEFLERFKSLLQKMIHQ |
| | | HLSSRTHGSEDSESKYGPPCPPCPAPEFLGGPSVF |
| | | LFPPKPKDTLMISRTPEVTCVVVDVSQEDPEVQFN |
| | | WYVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLTCLVKGFYPSDIAVEW |
| | | ESNGQPENNYKTTPPVLDSDGSFFLYSRLTVDKSR |
| | | WQEGNVFSCSVMHEALHNHYTQKSLSLSLGKMALI |
| | | VLGGVAGLLLFIGLGIFFCVRCRHRR |
| 12 | Membrane-bound IL-21GS18 | MWLQSLLLLGTVACSISHKSSSQGQDRHMIRMRQL |
| | amino acid sequence | IDIVDQLKNYVNDLVPEFLPAPEDVETNCEWSAFS |
| | | CFQKAQLKSANTGNNERIINVSIKKLKRKPPSTNA |
| | | GRRQKHRLTCPSCDSYEKKPPKEFLERFKSLLQKM |
| | | IHQHLSSRTHGSEDSGGGGSGGGGSGGGGSYFGKL |
| | | ESKLSVIRNLNDQVLFIDQGNRPLFEDMTDSDCRD |
| | | NAPRTIFIISMYKDSQPRGMAVTISVKCEKISTLS |
| | | CENKIISFKEMNPPDNIKDTKSDIIFFQRSVPGHD |
| | | NKMQFESSSYEGYFLACEKERDLFKLILKKEDELG |
| | | DRSIMFTVQNEDESKYGPPCPPCPAPEFLGGPSVF |
| | | LFPPKPKDTLMISRTPEVTCVVVDVSQEDPEVQFN |
| | | WYVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLTCLVKGFYPSDIAVEW |
| | | ESNGQPENNYKTTPPVLDSDGSFFLYSRLTVDKSR |
| | | WQEGNVFSCSVMHEALHNHYTQKSLSLSLGKMALI |
| | | VLGGVAGLLLFIGLGIFFCVRCRHRR |
| 13 | Membrane-bound IL-21GS7 | GCTAGCGCCACCATGTGGCTGCAGAGCCTGCTGCT |
| | nucleotide sequence | CTTGGGCACTGTGGCCTGCAGCATCTCTCACAAAT |
| | | CAAGCTCCCAAGGTCAAGATCGCCACATGATTAGA |
| | | ATGCGTCAACTTATAGATATTGTTGATCAGCTGAA |
| | | AAATTATGTGAATGACTTGGTCCCTGAATTTCTGC |
| | | CAGCTCCAGAAGATGTAGAGACAAACTGTGAGTGG |
| | | TCAGCTTTTTCCTGCTTTCAGAAGGCCCAACTAAA |
| | | GTCAGCAAATACAGGAAACAATGAAAGGATAATCA |
| | | ATGTATCAATTAAAAAGCTGAAGAGGAAACCACCT |
| | | TCCACAAATGCAGGGAGAAGACAGAAACACAGACT |
| | | AACATGCCCTTCATGTGATTCTTATGAGAAAAAAC |
| | | CACCCAAAGAATTCCTAGAAAGATTCAAATCACTT |
| | | CTCCAAAAGATGATTCATCAGCATCTGTCCTCTAG |
| | | AACACACGGAAGTGAAGATTCCGGCGGCGGCGGCA |
| | | GCGGCGGCGGCGGCAGCGGCGGCGGCGGCAGCGAT |
| | | TGCGATATCGAAGGCAAAGATGGTAAGCAGTACGA |
| | | AAGTGTTCTGATGGTTTCCATTGACCAACTGCTTG |
| | | ACTCCATGAAGGAAATTGGTTCTAATTGTCTTAAT |
| | | AATGAGTTTAACTTTTTTAAGAGACACATTTGCGA |
| | | TGCCAATAAGGAGGGAATGTTCCTTTTCCGCGCCG |
| | | CAAGAAAGCTGAGGCAATTCTTGAAGATGAACTCC |
| | | ACCGGCGATTTCGACTTGCACCTTTTGAAGGTCAG |
| | | CGAAGGAACGACCATTTTGCTGAACTGCACCGGCC |
| | | AGGTGAAGGGTCGGAAGCCAGCGGCACTGGGAGAG |
| | | GCGCAGCCGACTAAGAGCCTTGAGGAAAATAAGTC |
| | | TCTTAAAGAACAGAAAAAACTTAACGACCTGTGTT |
| | | TTCTGAAACGGTTGCTTCAGGAAATCAAGACCTGT |
| | | TGGAACAAAATACTGATGGGGACTAAAGAGCATGA |
| | | GTCCAAATATGGTCCCCCATGCCCACCATGCCCAG |
| | | CACCTGAGTTCCTGGGGGGACCATCAGTCTTCCTG |
| | | TTCCCCCCAAAACCCAAGGACACTCTCATGATCTC |
| | | CCGGACCCCTGAGGTCACGTGCGTGGTGGTGGACG |
| | | TGAGCCAGGAAGACCCCGAGGTCCAGTTCAACTGG |
| | | TACGTGGATGGCGTGGAGGTGCATAATGCCAAGAC |
| | | AAAGCCGCGGGAGGAGCAGTTCAACAGCACGTACC |
| | | GTGTGGTCAGCGTCCTCACCGTCCTGCACCAGGAC |
| | | TGGCTGAACGGTAAGGAGTACAAGTGCAAGGTCTC |
| | | CAACAAAGGCCTCCCGTCCTCCATCGAGAAAACCA |
| | | TCTCCAAAGCCAAAGGGCAGCCCCGAGAGCCACAG |
| | | GTGTACACCCTGCCCCCATCCCAGGAGGAGATGAC |
| | | CAAGAACCAGGTCAGCCTGACCTGCCTGGTCAAAG |
| | | GCTTCTACCCCAGCGACATCGCCGTGGAGTGGGAG |
| | | AGCAATGGGCAGCCGGAGAACAACTACAAGACCAC |
| | | GCCTCCCGTGCTGGACTCCGACGGCTCCTTCTTCC |
| | | TCTACAGCAGGCTAACCGTGGACAAGAGCAGGTGG |
| | | CAGGAGGGGAATGTCTTCTCATGCTCCGTGATGCA |
| | | TGAGGCTCTGCACAACCACTACACACAGAAGAGCC |
| | | TCTCCCTGTCTCTGGGTAAAATGGCCCTGATTGTG |
| | | CTGGGGGGCGTCGCCGGCCTCCTGCTTTTCATTGG |
| | | GCTAGGCATCTTCTTCTGTGTAAGGTGCAGGCACA |
| | | GGAGATAATAAGTCGAC |
| 14 | Membrane-bound IL-7GS21 | GCTAGCGCCACCATGTGGCTGCAGAGCCTGCTGCT |
| | nucleotide sequence | CTTGGGCACTGTGGCCTGCAGCATCTCTGATTGCG |
| | | ATATCGAAGGCAAAGATGGTAAGCAGTACGAAAGT |
| | | GTTCTGATGGTTTCCATTGACCAACTGCTTGACTC |
| | | CATGAAGGAAATTGGTTCTAATTGTCTTAATAATG |
| | | AGTTTAACTTTTTTAAGAGACACATTTGCGATGCC |
| | | AATAAGGAGGGAATGTTCCTTTTCCGCGCCGCAAG |
| | | AAAGCTGAGGCAATTCTTGAAGATGAACTCCACCG |
| | | GCGATTTCGACTTGCACCTTTTGAAGGTCAGCGAA |
| | | GGAACGACCATTTTGCTGAACTGCACCGGCCAGGT |
| | | GAAGGGTCGGAAGCCAGCGGCACTGGGAGAGGCGC |
| | | AGCCGACTAAGAGCCTTGAGGAAAATAAGTCTCTT |
| | | AAAGAACAGAAAAAACTTAACGACCTGTGTTTTCT |
| | | GAAACGGTTGCTTCAGGAAATCAAGACCTGTTGGA |
| | | ACAAAATACTGATGGGGACTAAAGAGCATGGCGGC |
| | | GGCGGCAGCGGCGGCGGCGGCAGCGGCGGCGGCGG |
| | | CAGCCACAAATCAAGCTCCCAAGGTCAAGATCGCC |
| | | ACATGATTAGAATGCGTCAACTTATAGATATTGTT |
| | | GATCAGCTGAAAAATTATGTGAATGACTTGGTCCC |
| | | TGAATTTCTGCCAGCTCCAGAAGATGTAGAGACAA |
| | | ACTGTGAGTGGTCAGCTTTTTCCTGCTTTCAGAAG |
| | | GCCCAACTAAAGTCAGCAAATACAGGAAACAATGA |
| | | AAGGATAATCAATGTATCAATTAAAAAGCTGAAGA |
| | | GGAAACCACCTTCCACAAATGCAGGGAGAAGACAG |
| | | AAACACAGACTAACATGCCCTTCATGTGATTCTTA |
| | | TGAGAAAAAACCACCCAAAGAATTCCTAGAAAGAT |
| | | TCAAATCACTTCTCCAAAAGATGATTCATCAGCAT |
| | | CTGTCCTCTAGAACACACGGAAGTGAAGATTCCGA |
| | | GTCCAAATATGGTCCCCCATGCCCACCATGCCCAG |
| | | CACCTGAGTTCCTGGGGGGACCATCAGTCTTCCTG |
| | | TTCCCCCCAAAACCCAAGGACACTCTCATGATCTC |
| | | CCGGACCCCTGAGGTCACGTGCGTGGTGGTGGACG |
| | | TGAGCCAGGAAGACCCCGAGGTCCAGTTCAACTGG |
| | | TACGTGGATGGCGTGGAGGTGCATAATGCCAAGAC |
| | | AAAGCCGCGGGAGGAGCAGTTCAACAGCACGTACC |
| | | GTGTGGTCAGCGTCCTCACCGTCCTGCACCAGGAC |
| | | TGGCTGAACGGTAAGGAGTACAAGTGCAAGGTCTC |
| | | CAACAAAGGCCTCCCGTCCTCCATCGAGAAAACCA |
| | | TCTCCAAAGCCAAAGGGCAGCCCCGAGAGCCACAG |
| | | GTGTACACCCTGCCCCCATCCCAGGAGGAGATGAC |
| | | CAAGAACCAGGTCAGCCTGACCTGCCTGGTCAAAG |
| | | GCTTCTACCCCAGCGACATCGCCGTGGAGTGGGAG |
| | | AGCAATGGGCAGCCGGAGAACAACTACAAGACCAC |
| | | GCCTCCCGTGCTGGACTCCGACGGCTCCTTCTTCC |
| | | TCTACAGCAGGCTAACCGTGGACAAGAGCAGGTGG |
| | | CAGGAGGGGAATGTCTTCTCATGCTCCGTGATGCA |
| | | TGAGGCTCTGCACAACCACTACACACAGAAGAGCC |
| | | TCTCCCTGTCTCTGGGTAAAATGGCCCTGATTGTG |
| | | CTGGGGGGCGTCGCCGGCCTCCTGCTTTTCATTGG |
| | | GCTAGGCATCTTCTTCTGTGTAAGGTGCAGGCACA |
| | | GGAGATAATAAGTCGAC |
| 15 | Membrane-bound IL-18GS21 | GCTAGCGCCACCATGTGGCTGCAGAGCCTGCTGCT |
| | nucleotide sequence | CTTGGGCACTGTGGCCTGCAGCATCTCTCACAAAT |
| | | CAAGCTCCCAAGGTCAAGATCGCCACATGATTAGA |
| | | ATGCGTCAACTTATAGATATTGTTGATCAGCTGAA |
| | | AAATTATGTGAATGACTTGGTCCCTGAATTTCTGC |
| | | CAGCTCCAGAAGATGTAGAGACAAACTGTGAGTGG |
| | | TCAGCTTTTTCCTGCTTTCAGAAGGCCCAACTAAA |
| | | GTCAGCAAATACAGGAAACAATGAAAGGATAATCA |
| | | ATGTATCAATTAAAAAGCTGAAGAGGAAACCACCT |
| | | TCCACAAATGCAGGGAGAAGACAGAAACACAGACT |
| | | AACATGCCCTTCATGTGATTCTTATGAGAAAAAAC |
| | | CACCCAAAGAATTCCTAGAAAGATTCAAATCACTT |
| | | CTCCAAAAGATGATTCATCAGCATCTGTCCTCTAG |
| | | AACACACGGAAGTGAAGATTCCGGCGGCGGCGGCA |
| | | GCGGCGGCGGCGGCAGCGGCGGCGGCGGCAGCGAT |
| | | TGCGATATCGAAGGCAAAGATGGTAAGCAGTACGA |
| | | AAGTGTTCTGATGGTTTCCATTGACCAACTGCTTG |
| | | ACTCCATGAAGGAAATTGGTTCTAATTGTCTTAAT |
| | | AATGAGTTTAACTTTTTTAAGAGACACATTTGCGA |
| | | TGCCAATAAGGAGGGAATGTTCCTTTTCCGCGCCG |
| | | CAAGAAAGCTGAGGCAATTCTTGAAGATGAACTCC |
| | | ACCGGCGATTTCGACTTGCACCTTTTGAAGGTCAG |
| | | CGAAGGAACGACCATTTTGCTGAACTGCACCGGCC |
| | | AGGTGAAGGGTCGGAAGCCAGCGGCACTGGGAGAG |
| | | GCGCAGCCGACTAAGAGCCTTGAGGAAAATAAGTC |
| | | TCTTAAAGAACAGAAAAAACTTAACGACCTGTGTT |
| | | TTCTGAAACGGTTGCTTCAGGAAATCAAGACCTGT |
| | | TGGAACAAAATACTGATGGGGACTAAAGAGCATGA |
| | | GTCCAAATATGGTCCCCCATGCCCACCATGCCCAG |
| | | CACCTGAGTTCCTGGGGGGACCATCAGTCTTCCTG |
| | | TTCCCCCCAAAACCCAAGGACACTCTCATGATCTC |
| | | CCGGACCCCTGAGGTCACGTGCGTGGTGGTGGACG |
| | | TGAGCCAGGAAGACCCCGAGGTCCAGTTCAACTGG |
| | | TACGTGGATGGCGTGGAGGTGCATAATGCCAAGAC |
| | | AAAGCCGCGGGAGGAGCAGTTCAACAGCACGTACC |
| | | GTGTGGTCAGCGTCCTCACCGTCCTGCACCAGGAC |
| | | TGGCTGAACGGTAAGGAGTACAAGTGCAAGGTCTC |
| | | CAACAAAGGCCTCCCGTCCTCCATCGAGAAAACCA |
| | | TCTCCAAAGCCAAAGGGCAGCCCCGAGAGCCACAG |
| | | GTGTACACCCTGCCCCCATCCCAGGAGGAGATGAC |
| | | CAAGAACCAGGTCAGCCTGACCTGCCTGGTCAAAG |
| | | GCTTCTACCCCAGCGACATCGCCGTGGAGTGGGAG |
| | | AGCAATGGGCAGCCGGAGAACAACTACAAGACCAC |
| | | GCCTCCCGTGCTGGACTCCGACGGCTCCTTCTTCC |
| | | TCTACAGCAGGCTAACCGTGGACAAGAGCAGGTGG |
| | | CAGGAGGGGAATGTCTTCTCATGCTCCGTGATGCA |
| | | TGAGGCTCTGCACAACCACTACACACAGAAGAGCC |
| | | TCTCCCTGTCTCTGGGTAAAATGGCCCTGATTGTG |
| | | CTGGGGGGCGTCGCCGGCCTCCTGCTTTTCATTGG |
| | | GCTAGGCATCTTCTTCTGTGTAAGGTGCAGGCACA |
| | | GGAGATAATAAGTCGAC |
| 16 | Membrane-bound IL-21GS18 | GCTAGCGCCACCATGTGGCTGCAGAGCCTGCTGCT |
| | nucleotide sequence | CTTGGGCACTGTGGCCTGCAGCATCTCTCACAAAT |
| | | CAAGCTCCCAAGGTCAAGATCGCCACATGATTAGA |
| | | ATGCGTCAACTTATAGATATTGTTGATCAGCTGAA |
| | | AAATTATGTGAATGACTTGGTCCCTGAATTTCTGC |
| | | CAGCTCCAGAAGATGTAGAGACAAACTGTGAGTGG |
| | | TCAGCTTTTTCCTGCTTTCAGAAGGCCCAACTAAA |
| | | GTCAGCAAATACAGGAAACAATGAAAGGATAATCA |
| | | ATGTATCAATTAAAAAGCTGAAGAGGAAACCACCT |
| | | TCCACAAATGCAGGGAGAAGACAGAAACACAGACT |
| | | AACATGCCCTTCATGTGATTCTTATGAGAAAAAAC |
| | | CACCCAAAGAATTCCTAGAAAGATTCAAATCACTT |
| | | CTCCAAAAGATGATTCATCAGCATCTGTCCTCTAG |
| | | AACACACGGAAGTGAAGATTCCGGCGGCGGCGGCA |
| | | GCGGCGGCGGCGGCAGCGGCGGCGGCGGCAGCTAC |
| | | TTTGGCAAGCTTGAATCTAAATTATCAGTCATAAG |
| | | AAATTTGAATGACCAAGTTCTCTTCATTGACCAAG |
| | | GAAATCGGCCTCTATTTGAAGATATGACTGATTCT |
| | | GACTGTAGAGATAATGCACCCCGGACCATATTTAT |
| | | TATAAGTATGTATAAAGATAGCCAGCCTAGAGGTA |
| | | TGGCTGTAACTATCTCTGTGAAGTGTGAGAAAATT |
| | | TCAACTCTCTCCTGTGAGAACAAAATTATTTCCTT |
| | | TAAGGAAATGAATCCTCCTGATAACATCAAGGATA |
| | | CAAAAAGTGACATCATATTCTTTCAGAGAAGTGTC |
| | | CCAGGACATGATAATAAGATGCAATTTGAATCTTC |
| | | ATCATACGAAGGATACTTTCTAGCTTGTGAAAAAG |
| | | AGAGAGACCTTTTTAAACTCATTTTGAAAAAAGAG |
| | | GATGAATTGGGGGATAGATCTATAATGTTCACTGT |
| | | TCAAAACGAAGACGAGTCCAAATATGGTCCCCCAT |
| | | GCCCACCATGCCCAGCACCTGAGTTCCTGGGGGGA |
| | | CCATCAGTCTTCCTGTTCCCCCCAAAACCCAAGGA |
| | | CACTCTCATGATCTCCCGGACCCCTGAGGTCACGT |
| | | GCGTGGTGGTGGACGTGAGCCAGGAAGACCCCGAG |
| | | GTCCAGTTCAACTGGTACGTGGATGGCGTGGAGGT |
| | | GCATAATGCCAAGACAAAGCCGCGGGAGGAGCAGT |
| | | TCAACAGCACGTACCGTGTGGTCAGCGTCCTCACC |
| | | GTCCTGCACCAGGACTGGCTGAACGGTAAGGAGTA |
| | | CAAGTGCAAGGTCTCCAACAAAGGCCTCCCGTCCT |
| | | CCATCGAGAAAACCATCTCCAAAGCCAAAGGGCAG |
| | | CCCCGAGAGCCACAGGTGTACACCCTGCCCCCATC |
| | | CCAGGAGGAGATGACCAAGAACCAGGTCAGCCTGA |
| | | CCTGCCTGGTCAAAGGCTTCTACCCCAGCGACATC |
| | | GCCGTGGAGTGGGAGAGCAATGGGCAGCCGGAGAA |
| | | CAACTACAAGACCACGCCTCCCGTGCTGGACTCCG |
| | | ACGGCTCCTTCTTCCTCTACAGCAGGCTAACCGTG |
| | | GACAAGAGCAGGTGGCAGGAGGGGAATGTCTTCTC |
| | | ATGCTCCGTGATGCATGAGGCTCTGCACAACCACT |
| | | ACACACAGAAGAGCCTCTCCCTGTCTCTGGGTAAA |
| | | ATGGCCCTGATTGTGCTGGGGGGCGTCGCCGGCCT |
| | | CCTGCTTTTCATTGGGCTAGGCATCTTCTTCTGTG |
| | | TAAGGTGCAGGCACAGGAGATAATAAGTCGAC |
| 17 | Mature human IL-21 | HKSSSQGQDRHMIRMRQLIDIVDQLKNYVNDLVPE |
| | variant 1 | FLPAPEDVETNCEWSAFSCFQKAQLKSANTGNNER |
| | amino acid sequence | IINVSIKKLKRKPPSTNAGRRQKHRLTCPSCDSYE |
| | | KKPPKEFLERFKSLLQKMIHQHLSSRTHGSEDS |
| 18 | Human IL-18 | YFGKLESKLSVIRNLNDQVLFIDQGNRPLFEDMTD |
| | amino acid | SDCRDNAPRTIFIISMYKDSQPRGMAVTISVKCEK |
| | sequence | ISTLSCENKIISFKEMNPPDNIKDTKSDIIFFQRS |
| | | VPGHDNKMQFESSSYEGYFLACEKERDLFKLILKK |
| | | EDELGDRSIMFTVQNED |
| 19 | GS Linker | GGGGSGGGGSGGGGS |
| 20 | IgG Linker | ESKYGPPCPPCPAPEFLGGPSVFLFPPKPKDTLMI |
| | | SRTPEVTCVVVDVSQEDPEVQFNWYVDGVEVHNAK |
| | | TKPREEQFNSTYRVVSVLTVLHQDWLNGKEYKCK |
| | | VSNKGLPSSIEKTISKAKGQPREPQVYTLPPSQEE |
| | | MTKNQVSLTCLVKGFYPSDIAVEWESNGQPENNYK |
| | | TTPPVLDSDGSFFLYSRLTVDKSRWQEGNVFSCSV |
| | | MHEALHNHYTQKSLSLSLGK |

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Design and Production of a Membrane-Bound IL-7

The present example describes design and production of membrane-bound IL-7 constructs. The membrane-bound IL-7 constructs contained the following features: a signal peptide; an IL-7 isoform; a linker; and a membrane-insertion moiety. For instance, an example of a membrane-bound IL-7 construct synthesized was one that included human GM-CSF signal peptide (SEQ ID NO: 1); mature human IL-7 variant 1 long isoform (SEQ ID NO: 2); a linker comprising an IgG4 hinge region (SEQ ID NO: 3), an IgG4 CH2 domain (SEQ ID NO: 4), and IgG4 CH3 domain (SEQ ID NO: 5); and a membrane-insertion moiety comprising Hu CD4 Tm Domain (SEQ ID NO: 6). An example of a membrane-bound IL-7 construct that was synthesized also includes membrane-bound IL-7 referred to as mIL-7 (SEQ ID NO: 7).

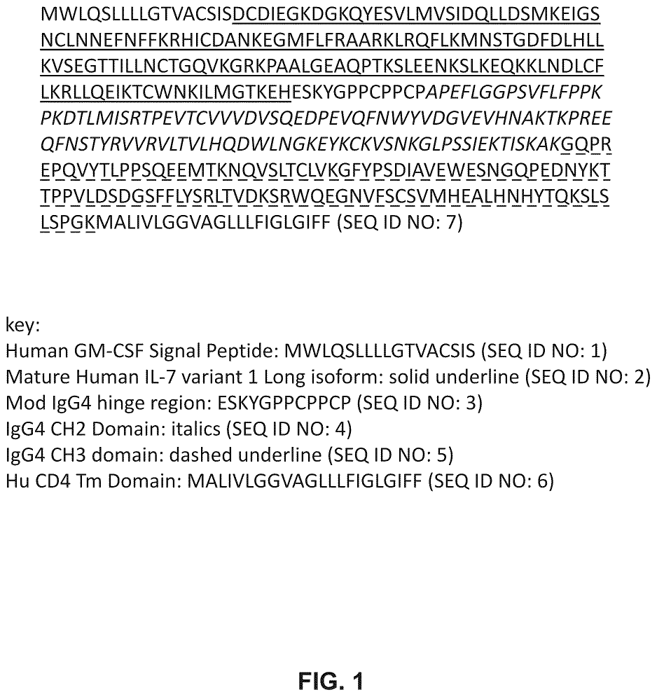

Referring now to FIG. 1, FIG. 1 presents a representation of the membrane bound IL-7 mIL-7 (SEQ ID NO: 7) comprising human GM-CSF signal peptide (SEQ ID NO: 1); mature human IL-7 variant 1 long isoform (SEQ ID NO: 2); a linker comprising an IgG4 hinge region (SEQ ID NO: 3), an IgG4 CH2 Domain (SEQ ID NO: 4), and IgG4 CH3 domain (SEQ ID NO: 5); and a membrane-insertion moiety comprising Hu CD4 Tm Domain (SEQ ID NO: 6).

Membrane-bound IL-7 was produced as follows. The mIL-7 construct was made as a gene block and cloned into pTRPE. Membrane particles were generated as described in Oyer, J. L., et al., Cytotherapy vol. 18,5 (2016): 653-63, which is hereby incorporated by reference in its entirety herein, with tangential flow filtration (TFF) used in addition to sucrose gradient centrifugation to purify the membrane particles (MPs).

### Example 2: Analysis of a Membrane-Bound IL-7

The membrane-bound IL-7 mIL-7 was subjected to analysis as described in the present example. In particular, expression tests and stimulation of T cells in relation to pSTAT5 and pSTAT3 signaling was analyzed.

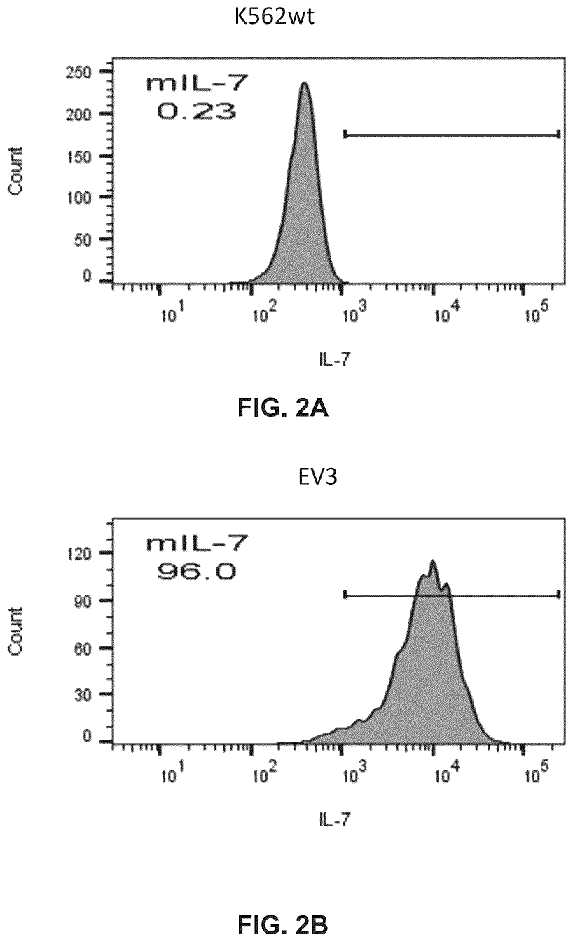

Referring now to FIG. 2A-FIG. 2B, FIG. 2A-FIG. 2B present graphical representations of results related to mIL-7 expression. Wildtype K562 and K562 cells expressing mIL-7 construct were stained with IL-7 antibody and expression of IL-7 was examined by flow cytometry. FIG. 2A presents a graphical representation of K562 wild type cells that were stained for IL-7. FIG. 2B presents a graphical representation of K562 cells transduced with mIL-7 that were stained for IL-7.

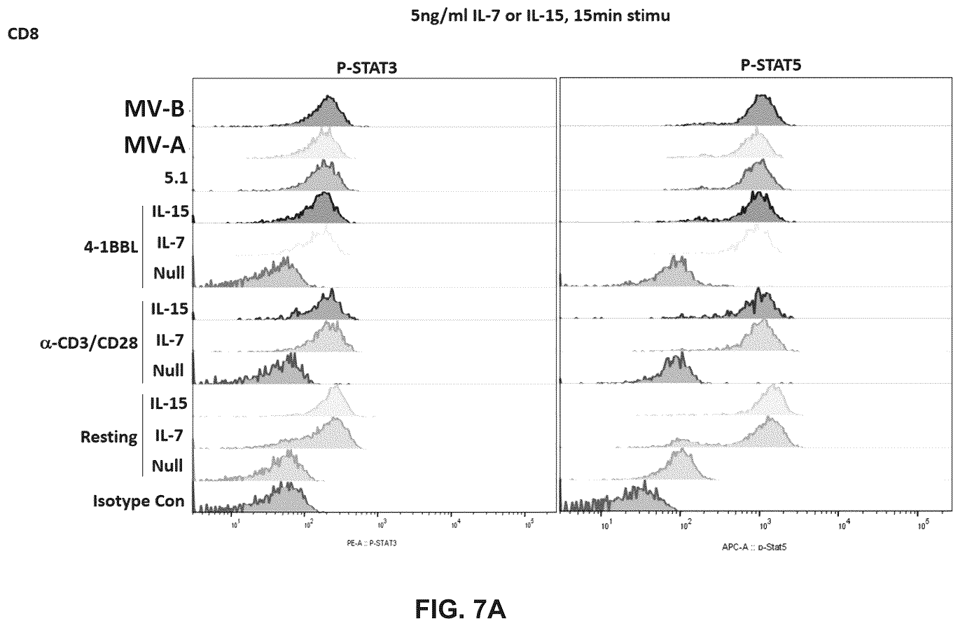

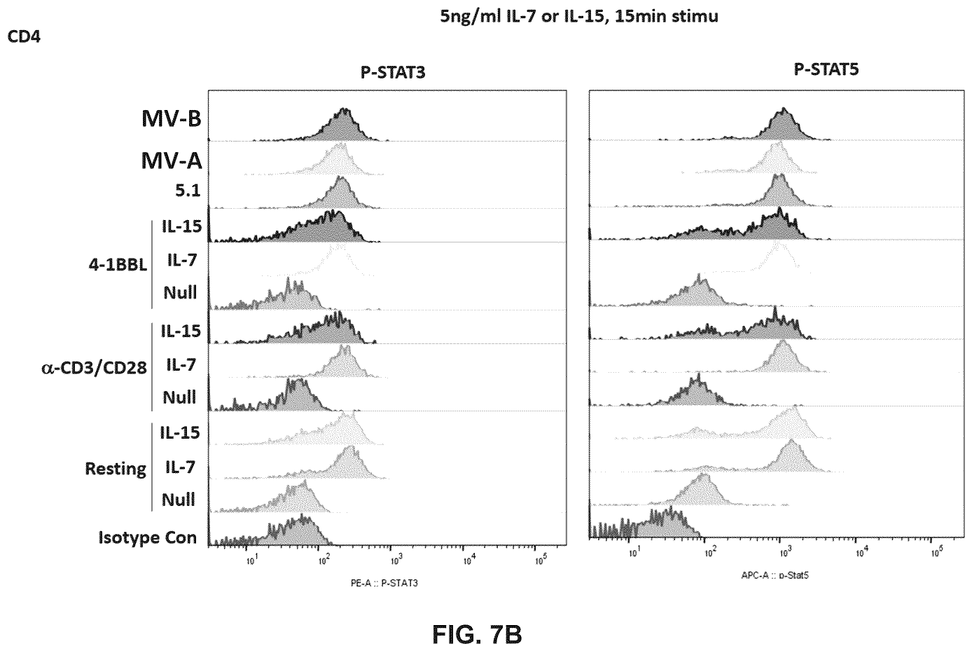

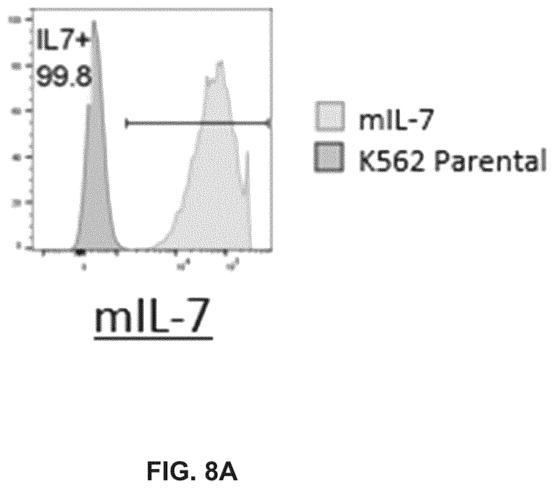

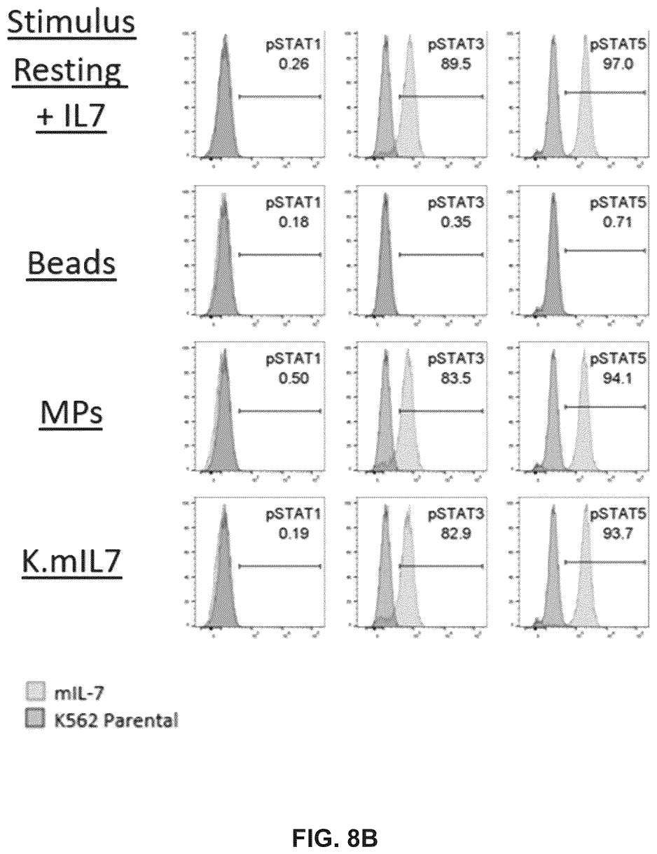

Referring now to FIG. 7A-FIG. 7B, FIG. 7A-FIG. 7B present graphical representations of pSTAT3 and pSTAT5 assays which analyzed the effects of mIL-7. Primary human T cells were either mixed with nothing (resting) or 5 ng/ml IL-7 or IL-15 ng/ml, CD3/28 coated beads alone (null), or 5 ng/ml IL-7 or IL-15 ng/ml, K.64.4-1BBL aAPCs loaded with anti-CD3 Ab alone or with 5 ng/ml IL-7 or IL-15 ng/ml, or K.64.86.4-1BBL IL-15/IL-15R and IL-7 loaded with anti-CD3 Ab, EV1, or EV for up to 30 min. pSTAT3 and pSTAT5 was measured using phosphorylation specific antibodies and flow cytometry. IL-15 and IL-7 were used as controls. FIG. 7A (CD8) and FIG. 7B (CD4) demonstrated the mIL-7 activated pSTAT3 and pSTAT5. In particular, EV1 and EV4 activated both STAT3 and STAT5, and the soluble cytokines likewise activated pSTAT3 and pSTAT5.

### Example 3: Design and Production of Dual Membrane-Bound Combokines

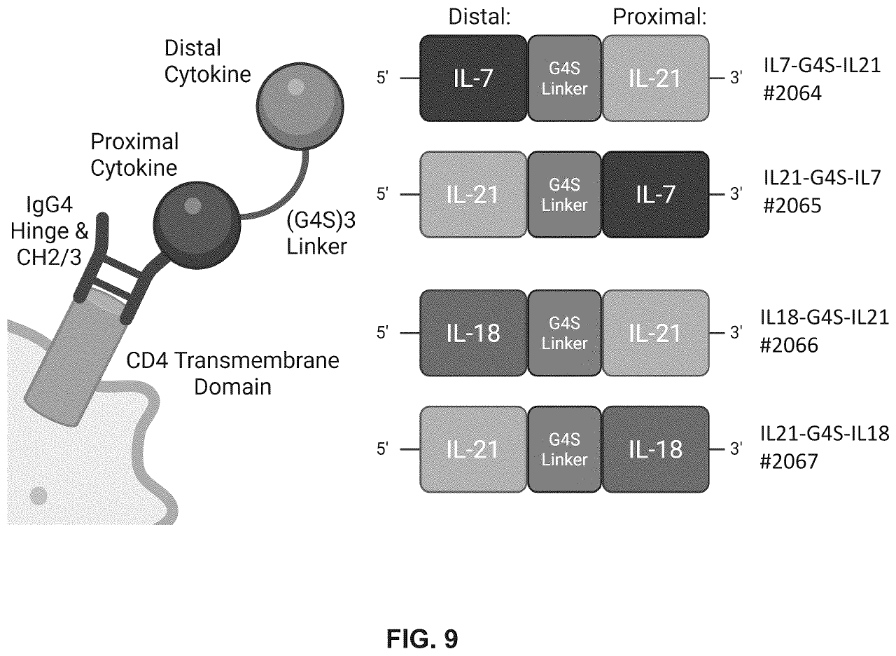

The present example describes the design and production of dual membrane-bound cytokines (combokines). In one embodiment, the dual membrane-bound cytokines include: a signal peptide, a first cytokine, a first linker, a second cytokine, a second linker, and a membrane-insertion moiety. In another embodiment, the combokine comprises a cytokine, a linker, a second cytokine, and a membrane-insertion moiety. Exemplary combokines as expressed on a cell surface are depicted in FIG. 9. In FIG. 9, the proximal cytokine is fused to the IgG4 hinge and CH2/3 IgG domains and CD4 transmembrane domain to enable membrane tethering.

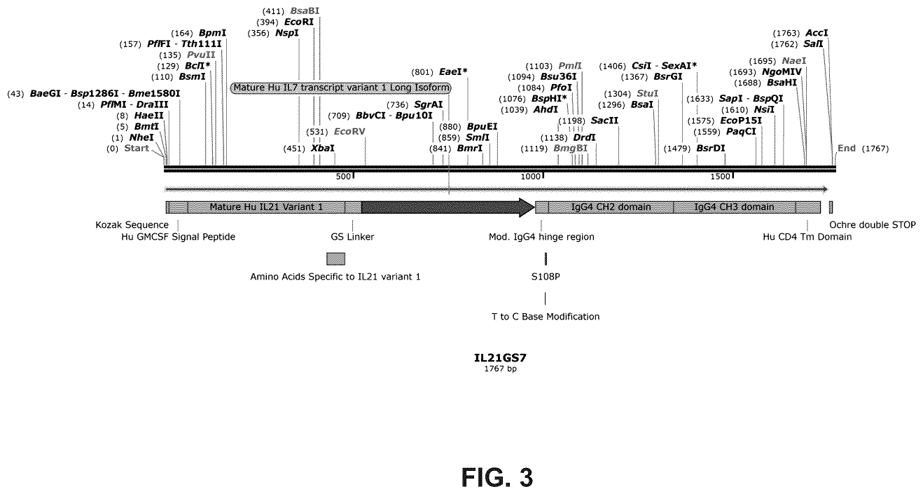

An example of a dual membrane bound cytokine construct includes one comprising human GM-CSF signal peptide (SEQ ID NO: 1); mature human IL-21 variant 1 (SEQ ID NO: 17); a GS linker (SEQ ID NO: 19); a mature human IL-7 variant 1 long isoform (SEQ ID NO: 2); a linker comprising an IgG4 hinge region (SEQ ID NO: 3), an IgG4 CH2 domain (SEQ ID NO: 4), and IgG4 CH3 domain (SEQ ID NO: 5); and a membrane-insertion moiety comprising Hu CD4 Tm domain (SEQ ID NO: 6). For instance, the dual membrane-bound cytokine comprising IL-21 and IL-7 comprises the amino acid sequence of SEQ ID NO: 9. The IL-21 comprises an S108P substitution in some iterations of such a dual membrane bound cytokine. A schematic representation of a dual membrane-bound cytokine comprising IL-21 and IL-7 is presented in FIG. 3.

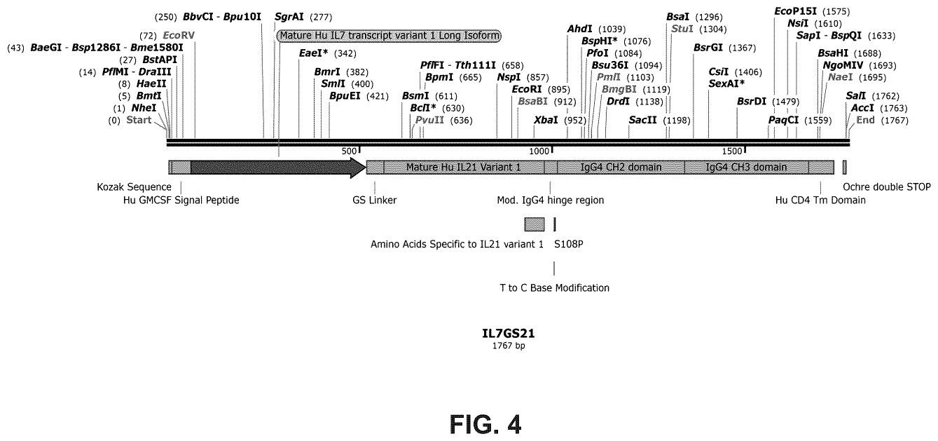

An example of a dual membrane bound cytokine construct includes one comprising human GM-CSF signal peptide (SEQ ID NO: 1); a mature human IL-7 variant 1 long isoform (SEQ ID NO: 2); a GS linker (SEQ ID NO: 19); mature human IL-21 variant 1 (SEQ ID NO: 17); a linker comprising an IgG4 hinge region (SEQ ID NO: 3), an IgG4 CH2 domain (SEQ ID NO: 4), and IgG4 CH3 domain (SEQ ID NO: 5); and a membrane-insertion moiety comprising Hu CD4 Tm Domain (SEQ ID NO: 6). For instance, the dual membrane-bound cytokine comprising IL-7 and IL-21 comprises the amino acid sequence of SEQ ID NO: 10. The IL-21 comprises an S108P substitution in some iterations of such a dual membrane bound cytokine. A schematic representation of a dual membrane-bound cytokine comprising IL-7 and IL-21 is presented in FIG. 4.

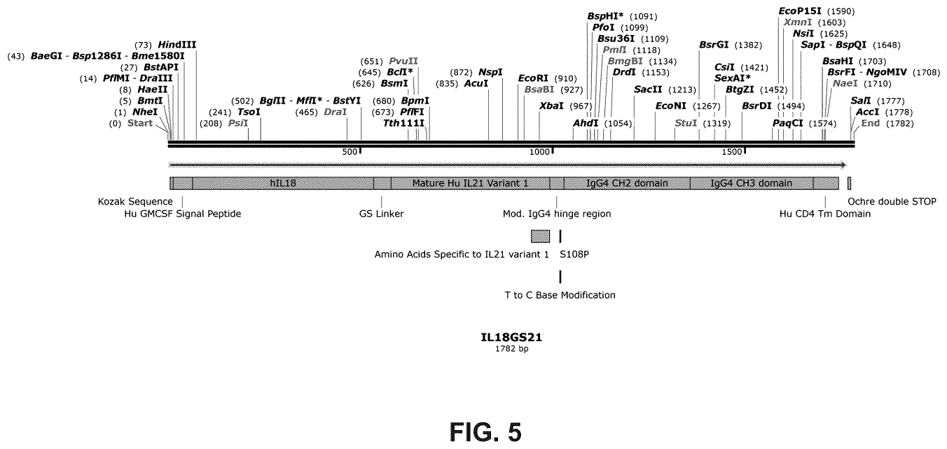

An example of a dual membrane bound cytokine construct includes one comprising human GM-CSF signal peptide (SEQ ID NO: 1); human IL-18 (SEQ ID NO: 18); a GS linker (SEQ ID NO: 19); mature human IL-21 variant 1 (SEQ ID NO: 17); a linker comprising an IgG4 hinge region (SEQ ID NO: 3), an IgG4 CH2 domain (SEQ ID NO: 4), and IgG4 CH3 domain (SEQ ID NO: 5); and a membrane-insertion moiety comprising Hu CD4 Tm Domain (SEQ ID NO: 6). For instance, the dual membrane-bound cytokine comprising IL-18 and IL-21 comprises the amino acid sequence of SEQ ID NO: 11. The IL-21 comprises an S108P substitution in some iterations of such a dual membrane bound cytokine. A schematic representation of a dual membrane-bound cytokine comprising IL-18 and IL-21 is presented in FIG. 5.

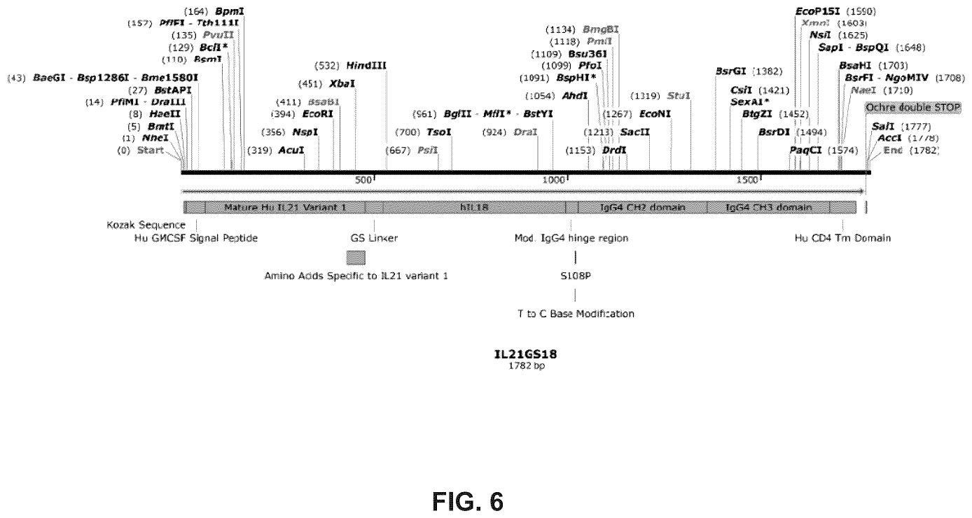

An example of a nucleic acid construct encoding a dual membrane bound cytokine includes one encoding: a human GM-CSF signal peptide (SEQ ID NO: 1); a human IL-21 variant 1 (SEQ ID NO: 17); a GS linker (SEQ ID NO: 19); a human IL-18 (SEQ ID NO: 18); a linker comprising an IgG4 hinge region (SEQ ID NO: 3), an IgG4 CH2 domain (SEQ ID NO: 4), and an IgG4 CH3 domain (SEQ ID NO: 5); and a membrane-insertion moiety comprising a human CD4 Tm Domain (SEQ ID NO: 6). For example, in one embodiment, a dual membrane-bound cytokine comprising IL-21 and IL-18 comprises the amino acid sequence of SEQ ID NO: 12. In some embodiments, the IL-21 comprises an S108P substitution. A schematic representation of a dual membrane-bound cytokine comprising IL-21 and IL-18 is presented in FIG. 6.

### Example 4: Expression of Combokines and Combokine-Directed Activation of STAT3, STAT5, and NF-κB Signaling

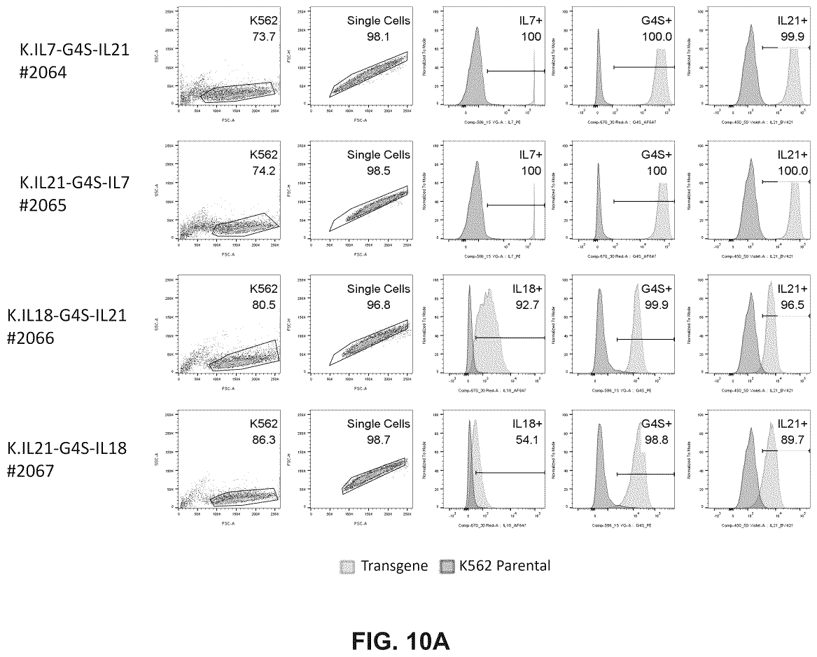

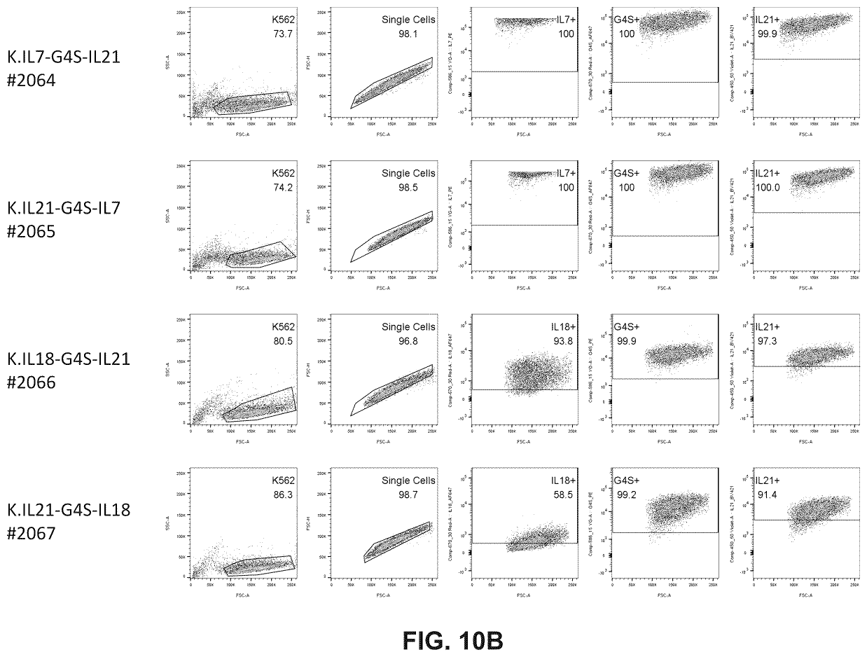

The combokines depicted in FIG. 9 were expressed in K562 cells and evaluated for activation of STAT3, STAT5, and NF-κB signaling under resting conditions or following stimulation with combokine-expressing K562 cells. Briefly, the K562 parental cell line (“K WT”) was transduced with recombinant combokine-expressing lentivirus constructs indicated viruses, initially expanded and passaged for 1-2 weeks to enrich for transduced cells, and sorted into single cells. Single clones were further cultured in Normocin-containing media for 3 weeks, and screened and selected for robust growth, highest transgene surface expression (IL-7, IL-18, and/or IL-21) and stimulatory function. FIGS. 10A and 10B present histogram (FIG. 10A) and dot plot (FIG. 10B) views showing expression of IL-7, IL-18, or IL-21 by fluorescence-activated cell sorting (FACS).

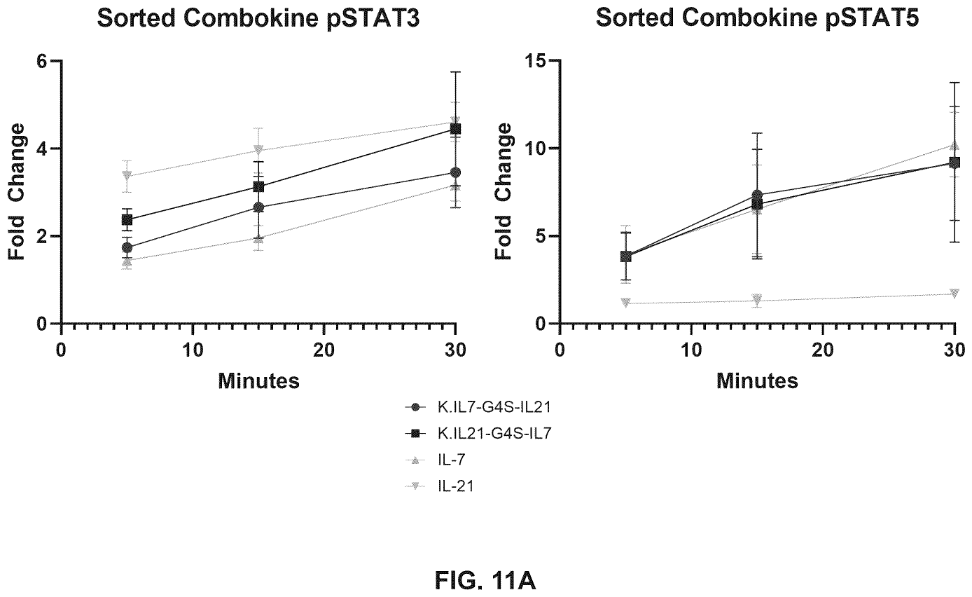

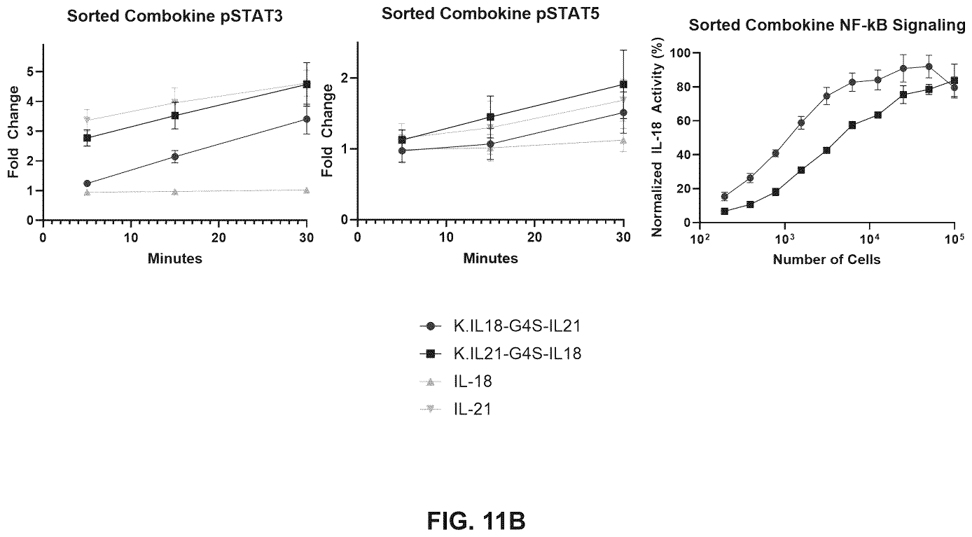

To evaluate the ability of combokine-transformed cells to promote expansion of T cells via activation of STAT3, STAT5, and/or NF-κB, 0.625e6 unstimulated cytotoxic T cells were stimulated with sorted single cell clones of K562 cells transformed with IL7-G4S-IL21 or IL-G4S-IL7 (FIG. 11A); or with IL18-G4S-IL21 or IL21-G4S-IL18 (FIG. 11B). In this case, the T cells were stimulated with the transformed K562 cells at 1:2 K:T ratio or with 10 ng/ml of IL-7 or IL-21 (FIG. 11A) or with 10 ng/mL of IL-18 or IL-21. Prior to stimulation, the T cells were stained with anti-CD4 and CD8 antibodies conjugated to fluorophores to prevent epitope masking during fixation following stimulation. Stimulated and fixed T cells were then washed, permeabilized and stained with pSTAT3 (left) and pSTAT5 (middle) antibodies. NF-κB activation (Right) is measured by incubating supernatant post-stimulation with an IL18-responsive HEK-Blue reporter cell line, with figures shown as a percentage of response induced by 0.2 ng soluble IL-18. pSTAT fold change calculated as MFI normalized to Resting condition. Data are expressed as mean±SEM from at least three independent donors and experiments.

The results in FIG. 11A indicate that between the IL-7 combokines (2064 and 2065), the IL-21 distal construct (i.e., IL21-G4S-IL7 #2065) induced higher pSTAT3, while pSTAT5 activity was comparable. The results in FIG. 11B indicate that while pSTAT5 activity is modest across the IL-18 combokines (2066 and 2067), the IL-21 distal construct (i.e., IL21-G4S-IL18 #2067) once again induces higher pSTAT3 activity but lower NF-κB signaling likely due to reduced IL-18 accessibility.

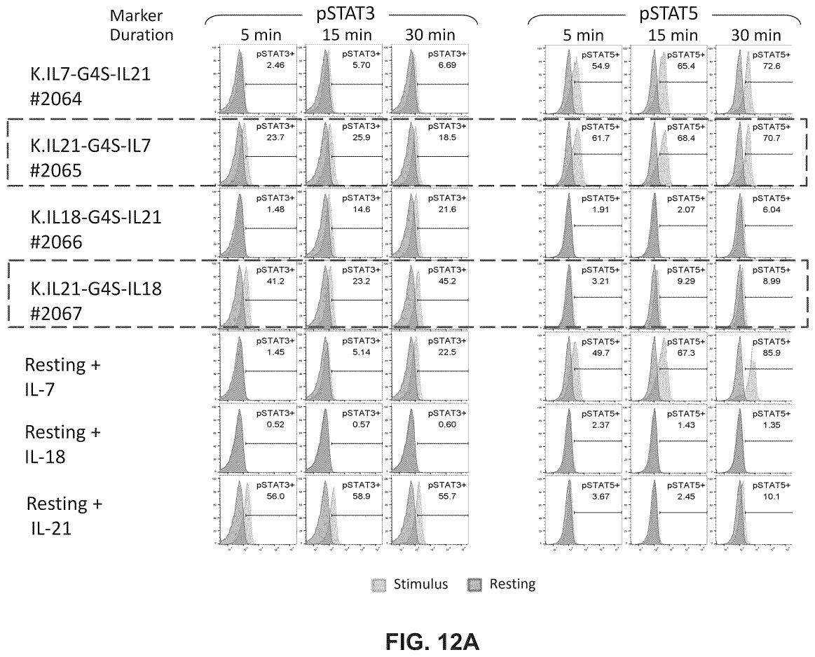

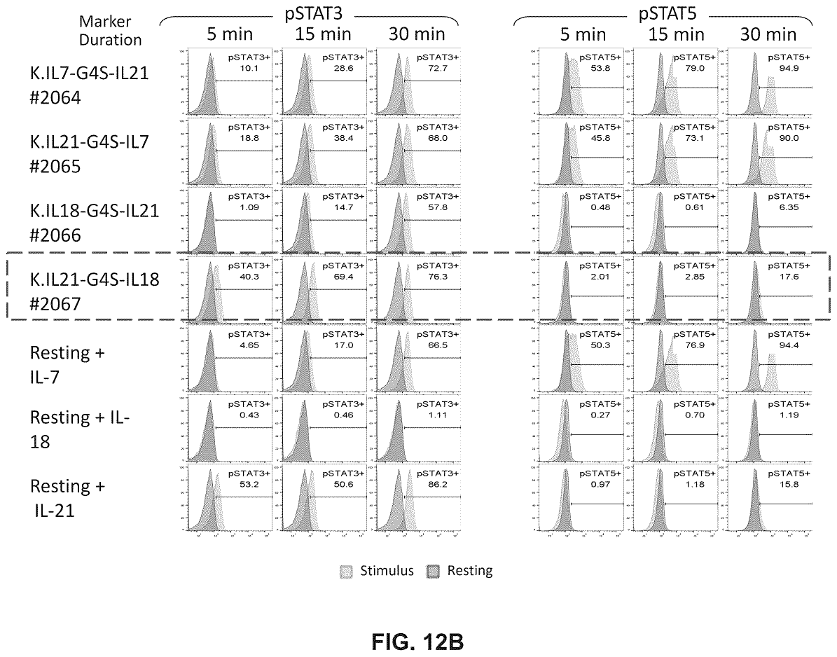

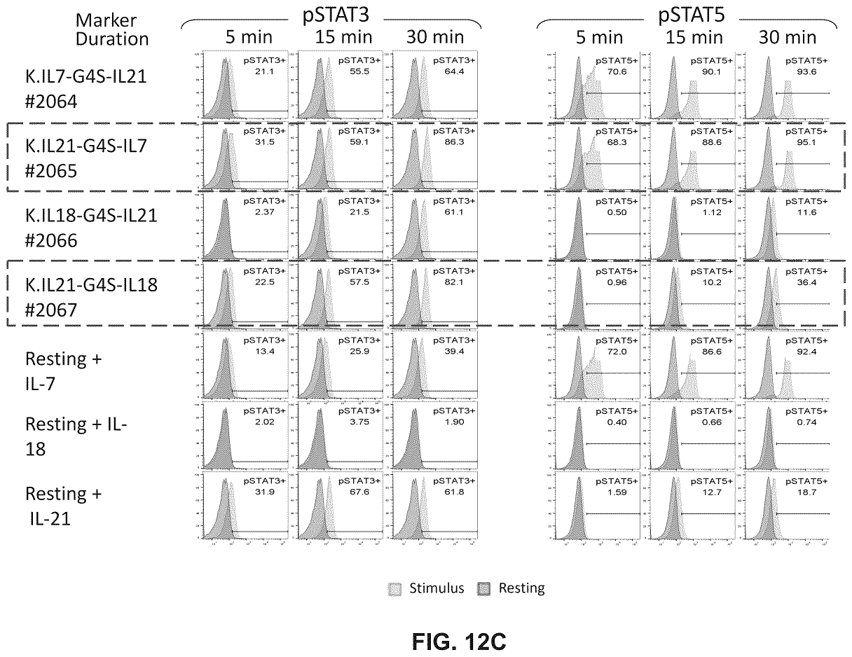

To further investigate the extent of STAT3 and STAT5 activation by the foregoing combokines as a function of time, 0.625e6 unstimulated cytotoxic T cells from each of three independent donors were stimulated with sorted single cell clones of K562 cells transformed with IL7-G4S-IL21, IL-G4S-IL7, IL18-G4S-IL21, or IL21-G4S-ILK18, or with IL-7, IL-18, IL-21. In this case, the T cells were stimulated with the transformed K562 cells at 1:2 K:T ratio or with 10 ng/ml of IL-7, IL-18, or IL-21 in 1.8 mL Eppendorf tubes for 5, 15, or 30 minutes. Prior to stimulation, the T cells were first stained with anti-CD4 and CD8 antibodies conjugated to fluorophores to prevent epitope masking during fixation following stimulation. Stimulated and fixed T cells were then washed, permeabilized and stained with pSTAT3 antibodies (left) or pSTAT5 antibodies (right). The results show activation of STAT3 (left) or STAT5 (right) in T cells from Donor 1 (FIG. 12A), Donor 2 (FIG. 12B), and Donor 3 (FIG. 12C). The results showed that the IL-21 distal combokines (i.e., IL21-G4S-IL7 #2065 and IL21-G4S-IL18 #2067) induce higher STAT3 phosphorylation than the IL-21 proximal combokines and that only the IL-7 expressing combokines induce meaningful STAT5 phosphorylation.

### Enumerated Embodiments

In some embodiments, the present invention is directed to the following non-limiting embodiments:

Embodiment 1 provides a membrane-bound interleukin-7 (IL-7) polypeptide comprising a signal sequence, an IL-7 amino acid sequence, a linker, and a membrane-insertion moiety.

Embodiment 2 provides the polypeptide of embodiment 1, wherein the IL-7 amino acid sequence comprises an amino acid sequence that is at least 95%, at least 96%, at least 97%, at least 98%, or at least 99% identical to the amino acid sequence of SEQ ID NO: 2.

Embodiment 3 provides the polypeptide of embodiment 3, wherein the IL-7 amino acid sequence comprises the amino acid sequence of SEQ ID NO: 2.

Embodiment 4 provides the polypeptide of any one of embodiments 1-3, wherein the linker comprises an amino acid sequence that is at least 95%, at least 96%, at least 97%, at least 98%, or at least 99% identical to the amino acid sequence of SEQ ID NO: 3, an amino acid sequence that is at least 95%, at least 96%, at least 97%, at least 98%, or at least 99% identical to the amino acid sequence of SEQ ID NO: 4, and/or an amino acid sequence that is at least 95%, at least 96%, at least 97%, at least 98%, or at least 99% identical to the amino acid sequence of SEQ ID NO: 5.

Embodiment 5 provides the polypeptide of embodiment 4, wherein the linker comprises the amino acid sequence of SEQ ID NO: 3, the amino acid sequence of SEQ ID NO: 4, and/or the amino acid sequence of SEQ ID NO: 5.

Embodiment 6 provides the polypeptide of embodiment 4, wherein the linker comprises, from N-terminus to C-terminus, an amino acid sequence that is at least 95%, at least 96%, at least 97%, at least 98%, or at least 99% identical to the amino acid sequence of SEQ ID NO: 3, an amino acid sequence that is at least 95%, at least 96%, at least 97%, at least 98%, or at least 99% identical to the amino acid sequence of SEQ ID NO: 4, and an amino acid sequence that is at least 95%, at least 96%, at least 97%, at least 98%, or at least 99% identical to the amino acid sequence of SEQ ID NO: 5

Embodiment 7 provides the polypeptide of embodiment 6, wherein the linker comprises, from N-terminus to C-terminus, the amino acid sequence of SEQ ID NO: 3, the amino acid sequence of SEQ ID NO: 4, and the amino acid sequence of SEQ ID NO: 5.

Embodiment 8 provides the polypeptide of embodiment 7, wherein the linker comprises an amino acid sequence that is at least 95%, at least 96%, at least 97%, at least 98%, or at least 99% identical to the amino acid sequence of SEQ ID NO: 20.

Embodiment 9 provides the polypeptide of embodiment 7, wherein the linker comprises the amino acid sequence of SEQ ID NO: 20.

Embodiment 10 provides the polypeptide of any one of the embodiments 1-9, wherein the membrane-insertion moiety comprises an amino acid sequence that is at least 95%, at least 96%, at least 97%, at least 98%, or at least 99% identical to SEQ ID NO: 6.

Embodiment 11 provides the polypeptide of embodiment 10, wherein the membrane-insertion moiety comprises the amino acid sequence of SEQ ID NO: 6.

Embodiment 12 provides the polypeptide of any one of embodiments 1-11, comprising an amino acid sequence at least 95%, at least 96%, at least 97%, at least 98%, or at least 99% identical to the amino acid sequence of SEQ ID NO: 7.

Embodiment 13 provides the polypeptide of embodiment 12, comprising the amino acid sequence of SEQ ID NO: 7.

Embodiment 14 provides the polypeptide of any one of embodiments 1-13, wherein the polypeptide is capable of activating pSTAT3 and/or pSTAT5 signaling in a cell.

Embodiment 15 provides the polypeptide of embodiment 14, wherein the cell is a T-cell.

Embodiment 16 provides the polypeptide of any one of embodiments 1-15, wherein the polypeptide further comprises IL-21.

Embodiment 17 provides the polypeptide of embodiment 16, wherein the IL-21 comprises an S108P substitution.

Embodiment 18 provides the polypeptide of embodiment 16 or 17, wherein the polypeptide comprises an amino acid sequence at least 95%, at least 96%, at least 97%, at least 98%, or at least 99% identical to SEQ ID NO: 9.

Embodiment 19 provides the polypeptide of embodiment 18, wherein the polypeptide comprises the amino acid sequence of SEQ ID NO: 9.

Embodiment 20 provides the polypeptide of embodiment 16 or 17, wherein the polypeptide comprises an amino acid sequence at least 95%, at least 96%, at least 97%, at least 98%, or at least 99% identical to SEQ ID NO: 10.

Embodiment 21 provides the polypeptide of embodiment 20, wherein the polypeptide comprises the amino acid sequence of SEQ ID NO: 10.

Embodiment 22 provides a membrane bound polypeptide comprising, from amino terminus to carboxy terminus, a first cytokine, linker, a second cytokine, and a membrane-insertion moiety, wherein the first cytokine comprises IL-21 and the second cytokine comprises IL-7, or wherein the first cytokine comprises IL-7 and the second cytokine comprises IL-21.

Embodiment 23 provides the polypeptide of embodiment 22, wherein IL-7 comprises an amino acid sequence that is at least 95%, at least 96%, at least 97%, at least 98%, or at least 99% identical to the amino acid sequence of SEQ ID NO: 2.

Embodiment 24 provides the polypeptide of embodiment 23, wherein the IL-7 amino acid sequence comprises the amino acid sequence of SEQ ID NO: 2.

Embodiment 25 provides the polypeptide of any one of embodiments 22-24, wherein the first cytokine comprises IL-21 and the second cytokine comprises IL-7.

Embodiment 26 provides the polypeptide of embodiment 25, wherein the polypeptide comprises an amino acid sequence that is at least 95%, at least 96%, at least 97%, at least 98%, or at least 99% identical to the amino acid sequence of SEQ ID NO: 9.

Embodiment 27 provides the polypeptide of embodiment 26, wherein the polypeptide comprises the amino of SEQ ID NO: 9.

Embodiment 28 provides the polypeptide of any one of embodiments 22-24, wherein the first cytokine comprises IL-7 and the second cytokine comprises IL-21.

Embodiment 29 provides the polypeptide of embodiment 28, comprising an amino acid sequence that is at least 95%, at least 96%, at least 97%, at least 98%, or at least 99% identical to the amino acid sequence of SEQ ID NO: 10.

Embodiment 30 provides the polypeptide of embodiment 29, comprising the amino acid sequence of SEQ ID NO: 10.

Embodiment 31 provides a membrane bound polypeptide comprising, from amino terminus to carboxy terminus, a first cytokine, linker, a second cytokine, and a membrane-insertion moiety, wherein the first cytokine comprises IL-18 and the second cytokine comprises IL-21, or wherein the first cytokine comprises IL-21 and the second cytokine comprises IL-18.

Embodiment 32 provides the polypeptide of embodiment 31, wherein the first cytokine comprises IL-18 and the second cytokine comprises IL-21.

Embodiment 33 provides the polypeptide of embodiment 32, wherein the polypeptide comprises an amino acid sequence that is at least 95%, at least 96%, at least 97%, at least 98%, or at least 99% identical to the amino acid sequence of SEQ ID NO: 11.

Embodiment 34 provides the polypeptide of embodiment 33, wherein the polypeptide comprises the amino acid sequence of SEQ ID NO: 11.

Embodiment 35 provides the polypeptide of embodiment 31, wherein the first cytokine comprises IL-21 and the second cytokine comprises IL-18.

Embodiment 36 provides the polypeptide of embodiment 35, wherein the polypeptide comprises an amino acid sequence that is at least 95%, at least 96%, at least 97%, at least 98%, or at least 99% identical to the amino acid sequence of SEQ ID NO: 12.

Embodiment 37 provides the polypeptide of embodiment 35, wherein the polypeptide comprises the amino acid sequence of SEQ ID NO: 12.

Embodiment 38 provides a polynucleotide encoding the polypeptide of any one of embodiments 1-37.

Embodiment 39 provides the polynucleotide of embodiment 38, comprising a signal sequence encoding an amino acid sequence that is at least 95%, at least 96%, at least 97%, at least 98%, or at least 99% identical to the amino acid sequence of SEQ ID NO: 1.

Embodiment 40 provides the polynucleotide of embodiment 39, wherein the signal sequence encodes the amino acid sequence of SEQ ID NO 1.

Embodiment 41 provides a vector comprising the polynucleotide of any one of embodiments 38-40.

Embodiment 42 provides the vector of embodiment 41, wherein the vector is an expression vector.

Embodiment 43 provides an isolated host cell comprising the polypeptide of any one of embodiments 1-37, the polynucleotide of any one of embodiments 38-40, or the vector of embodiment 41 or 42.

Embodiment 44 provides a membrane vesicle comprising the polypeptide of any one of embodiments 1-37.

Embodiment 45 provides a pharmaceutical composition comprising a therapeutically effective amount of the polypeptide of any one of embodiments 1-37 or the membrane vesicle of embodiment 44; and a pharmaceutically acceptable carrier.

Embodiment 46 provides a method for stimulating and expanding a T cell, comprising contacting the T cell with the polypeptide of any one of embodiments 1-37, the membrane vesicle of embodiment 44, or the pharmaceutical composition of embodiment 45.

Embodiment 47 provides the method of embodiment 46, wherein the T cell is an autologous T cell.

Embodiment 48 provides the method of embodiment 46 or 47, wherein the T cell is a human T cell.

Embodiment 49 provides a method for stimulating and expanding a regulatory T cell (Treg), comprising contacting the Treg with the polypeptide of any one of embodiments 1-37, the membrane vesicle of embodiment 44, or the pharmaceutical composition of embodiment 45.

Embodiment 50 provides the method of embodiment 49, wherein the Treg is a human T cell.

Embodiment 51 provides the method of embodiment 50, wherein the Treg is a non-human primate T cell.

Embodiment 52 provides the method of embodiment 51, wherein the non-human primate is Macaca fascicularis.

Embodiment 53 provides the method of embodiment 51, wherein the non-human primate is Macaca mulatta.

The foregoing outlines features of several embodiments so that those skilled in the art may better understand the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

In sum, while this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A membrane-bound interleukin-7 (IL-7) polypeptide comprising an IL-7 amino acid sequence, a linker, and a membrane-insertion moiety.

2. The polypeptide of any one of claim 1, wherein the IL-7 amino acid sequence comprises an amino acid sequence that is at least 95% identical to the amino acid sequence of SEQ ID NO: 2.

3. The polypeptide of claim 1 or 2, wherein the linker comprises, from N-terminus to C-terminus, an amino acid sequence that is at least 95% identical to the amino acid sequence of SEQ ID NO: 3, an amino acid sequence that is at least 95% identical to the amino acid sequence of SEQ ID NO: 4, and an amino acid sequence that is at least 95% identical to the amino acid sequence of SEQ ID NO: 5.

4. The polypeptide of claim 3, wherein the linker comprises an amino acid sequence that is at least 95% identical to the amino acid sequence of SEQ ID NO: 20.

5. The polypeptide of any one of the claims 1-4, wherein the membrane-insertion moiety comprises an amino acid sequence that is at least 95% identical to SEQ ID NO: 6.

6. The polypeptide of any one of claim 1-5, comprising an amino acid sequence at least 95% identical to the amino acid sequence of SEQ ID NO: 7.

7. The polypeptide of any one of claims 1-6, wherein the polypeptide further comprises IL-21.

8. The polypeptide of claim 7, wherein the polypeptide comprises an amino acid sequence at least 95% identical to SEQ ID NO: 9 or SEQ ID NO: 10.

9. A membrane bound polypeptide comprising, from amino terminus to carboxy terminus, a first cytokine, a linker, a second cytokine, and a membrane-insertion moiety, wherein the first cytokine comprises IL-21 and the second cytokine comprises IL-7, or wherein the first cytokine comprises IL-7 and the second cytokine comprises IL-21.

10. The polypeptide of claim 9, wherein IL-7 comprises an amino acid sequence that is at least 95% identical to the amino acid sequence of SEQ ID NO: 2.

11. The polypeptide of claim 9 or 10, wherein the first cytokine comprises IL-21 and the second cytokine comprises IL-7.

12. The polypeptide of claim 11, wherein the polypeptide comprises an amino acid sequence that is at least 95% identical to the amino acid sequence of SEQ ID NO: 9.

13. The polypeptide of claim 9 or 10, wherein the first cytokine comprises IL-7 and the second cytokine comprises IL-21.

14. The polypeptide of claim 13, comprising an amino acid sequence that is at least 95% identical to the amino acid sequence of SEQ ID NO: 10.

15. A membrane bound polypeptide comprising, from amino terminus to carboxy terminus, a first cytokine, a linker, a second cytokine, and a membrane-insertion moiety, wherein the first cytokine comprises IL-18 and the second cytokine comprises IL-21, or wherein the first cytokine comprises IL-21 and the second cytokine comprises IL-18.

16. The polypeptide of claim 15, wherein the first cytokine comprises IL-18 and the second cytokine comprises IL-21.

17. The polypeptide of claim 16, wherein the polypeptide comprises an amino acid sequence that is at least 95% identical to the amino acid sequence of SEQ ID NO: 11.

18. The polypeptide of claim 15, wherein the first cytokine comprises IL-21 and the second cytokine comprises IL-18.

19. The polypeptide of claim 18, wherein the polypeptide comprises an amino acid sequence that is at least 95% identical to the amino acid sequence of SEQ ID NO: 12.

20. An isolated polynucleotide encoding the polypeptide of any one of claims 1-19.

21. The polynucleotide of claim 20, comprising a signal sequence encoding an amino acid sequence that is at least 95% identical to the amino acid sequence of SEQ ID NO: 1.

22. A vector comprising the polynucleotide of claim 20 or 21.

23. The vector of claim 22, wherein the vector is an expression vector.

24. An isolated host cell comprising the polypeptide of any one of claims 1-19, the polynucleotide of claims 20 or 21, or the vector of claim 22 or 23.

25. A membrane vesicle comprising the polypeptide of any one of claims 1-19.

26. A pharmaceutical composition comprising a therapeutically effective amount of the polypeptide of any one of claims 1-19 or the membrane vesicle of claim 44; and a pharmaceutically acceptable carrier.

27. A method for stimulating and expanding a T cell, comprising contacting the T cell with the polypeptide of any one of claims 1-19, the membrane vesicle of claim 25, or the pharmaceutical composition of claim 26.

28. The method of claim 46, wherein the T cell is an autologous T cell.

29. A method for stimulating and expanding a regulatory T cell (Treg), comprising contacting the Treg with the polypeptide of any one of claims 1-19, the membrane vesicle of claim 25, or the pharmaceutical composition of claim 26.

30. The method of claim 29, wherein the Treg is a human T cell or a non-human primate T cell.
